(19) United States
(12) Patent Application Publication
(10) Pub. No.: US 2024/0252580 A1
(43) Pub. Date: Aug. 1, 2024

(54) AMINO ACID HYDRATION FORMULATION AND METHOD OF USE

(71) Applicant: The Coca-Cola Company, Atlanta, GA (US)

(72) Inventors: Haiyu REN, Atlanta, GA (US); Yu SHI, Atlanta, GA (US); Nilesh Bansilal KARAVA, Atlanta, GA (US); Dattatreya GAJULA, Murietta, GA (US)

(21) Appl. No.: 18/565,042

(22) PCT Filed: May 27, 2022
(86) PCT No.: PCT/US2022/031330

§ 371 Date: Nov. 28, 2023

Related U.S. Application Data
(60) Provisional application No. 63/194,567, filed on May 28, 2021.

(51) Int. Cl. A61K 38/05; A61K 9/00; A61K 31/198; A61K 31/7004

(52) U.S. Cl. CPC A61K 38/05; A61K 9/0095; A61K 31/198; A61K 31/7004

(57) ABSTRACT

The present disclosure presents compositions and methods for hydration or rehydration. A composition for oral consumption comprises an amino acid formulation. A method of hydrating or rehydrating a human, the method comprising administering an effective amount of an oral composition, wherein the oral composition comprises an amino acid formulation. The amino acid formulation may include an amino acid, a dipeptide, an oligopeptide, a carbohydrate-amino acid complex, or any combinations thereof.

This application is being filed on May 27, 2022, as a PCT International Patent application and claims the benefit of and priority to U.S. Provisional patent application Ser. No. 63/194,567, filed May 28, 2021, the entire disclosure of which is incorporated by reference herein in its entirety.

### INTRODUCTION

The present disclosure generally relates to compositions and methods for hydration or rehydration.

Humans often suffer from dehydration or hypohydration under stressful conditions such as exposure to excess heat, heavy or extended periods of exercise, demanding physical activity with limited water, high-mountain climbing, or during extended water loss from diarrhea, vomiting or sweating. Negative physiological effects such as loss of electrolytes balances often accompanied with dehydration. It would be beneficial to provide a composition for rapid and effective hydration or rehydration, which could restore body fluid and critical elements to maintain performance and avoid any potential adverse health issues with dehydration.

Attempts have been made previously to prepare rehydration compositions and in particular, beverages and sport drinks, which provide the energy source, electrolytes, and water for rehydration. These beverages may contain mixtures of proteins, sugars such as glucose, fructose, maltose, salts, and other additives such as citric acid, glycerol, triacylgylcerol, sodium acid sulfate, which are alleged to be beneficial for rehydration. These beverages and compositions are generally disclosed, for examples, in U.S. Pat. Nos. 4,853,237, 5,447,730, 6,221,910, 6,485,764, 7,001,612, 7,160,565, 8,993,032, and U.S. Pat. App. Nos. 2005/0100637, 2005/0048136, 2009/0117224, 2012/0128815.

However, many disclosed compositions could be more effective. For example, many compositions do not provide rapid restoration of body fluid and plasma volume, and the duration for rehydration effect is often relatively short. In particular, the retention of body fluid after ingesting these compositions may not last long, and the total body fluid decreases quickly to the level prior to rehydration. Additionally, the effective component of the rehydrating composition and/or the direct evidence of rehydration effect am not clearly indicated in prior disclosures. Moreover, many existing compositions are high in carbohydrates, and/or other high calorie ingredients that may be less preferred by consumers. There is also trade-off between the goal of rapid fluid restoration and the goal of energy supplementation when a person consumes a carbohydrate-containing drink. As the carbohydrate concentration of a drink increases, its energy density and osmolality increases, thereby reducing its gastric emptying rate. Consequently, the effectiveness of fluid restoration is undermined.

Therefore, in spite of the above disclosures, it is still highly desirable for new hydrating or rehydrating compositions and beverages that provide a rapid and effective solution to dehydration and hypohydration, long duration of hydration or rehydration, compensation and extended period of retention of total body fluid, extended restoration of electrolyte balance, and/or improved taste and palatability profile.

### SUMMARY OF DISCLOSURE

The present disclosure presents hydrating and rehydrating compositions and methods that meet the above stated needs.

In one aspect, the present disclosure relates to a composition for oral consumption comprising an amino acid formulation. The amino acid formulation may include an amino acid, a dipeptide, an oligopeptide, a carbohydrate-amino acid complex, or any combinations thereof. In one particular embodiment, the amino acid formulation consists essentially of glycine, or a glycine-containing dipeptide, or a carbohydrate-glycine complex, or any combinations thereof. In some embodiments, the amino acid formulation or the oral composition is free or substantially free from a branched-chain amino acid (BCAA).

In some embodiments, the oral composition is a ready-to-drink hydrating beverage. One particular example is a sport beverage or sport drink. In some embodiments, the beverage has a concentration of the amino acid formulation from about 1 g/L to about 50 g/L, or from about 2 g/L to about 30 g/L, or from about 3 g/L to about 30 g/L, or from about 4 g/L to about 20 g/L, or from about 5 g/L to about 10 g/L, 30 or from about 0.05 g/L to about 1 g/L based on the total volume of the beverage.

In some embodiments, the oral composition is in a dry or semi-dry form. One particular example is a dry powder that is readily soluble in water. In some embodiments, a drinkable solution of the dry powder can be readily prepared by dissolving the dry powder in a drinkable medium, wherein the solution has a concentration of the amino acid formulation from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L based on the total volume of the drinkable solution.

In some embodiments, the present composition further comprises at least one electrolyte. Examples of electrolyte include sodium, potassium, calcium, magnesium, chloride, phosphate, bicarbonate, and combinations thereof.

In some embodiments, the present composition further comprises at least one sweetener. The sweetener can be a carbohydrate, a peptide-based sweetener, a non-nutritive sweetener, an artificial sweetener, a commercial sweetener or sweetening composition, or a “natural high potency sweetener” (NHPS). In one particular embodiment, the present composition comprises a low- or non-calorie sweetener. In another particular embodiment, the present composition is free of substantially free from a carbohydrate sweetener.

In some embodiments, the present composition further include at least one additive, at least one functional ingredient, or both.

In some embodiments, the present composition has an osmolality from about 250 to about 350 mOsm/kg, or from about 260 to about 340 mOsm/kg, or from about 270 to about 330 mOsm/kg, or from about 280 to about 320 mOsm/kg, or from about 290 to about 310 mOsm/kg, or from about 290 to about 300 mOsm/kg.

In another aspect, the present disclosure relates to a method for hydration or rehydration. The present method utilizes the compositions described herein for at least one of the following purposes: providing a rapid impact on plasma volume (i.e. D₂O or water) restoration during rehydration, attenuating or reversing the effects of dehydration or hypohydration, ameliorating other adverse effects of exercise, heat or other activity which causes bodily fluid loss, providing a positive impact on subsequent physical performance, enhancing the duration of body fluid retention, rapidly increasing plasma volume, maintaining the increased plasma volume for long duration, restoring and maintaining electrolyte balance, providing energy source, balancing or controlling calorie uptake, and stimulating thirst and drinking.

In some embodiments, the present disclosure provides a method of hydrating or rehydrating a human, the method comprising administering an effective amount of the oral composition described herein, wherein the oral composition comprises an amino acid formulation described herein.

In some embodiments of the present method, the oral composition is a ready-to-drink hydrating beverage, or a sports drink, or an enhanced water drink. In some embodiments, the concentration of the amino acid formulation is from about 1 g/L to about 50 g/L, or from about 2 g/L to about 30 g/L, or from about 3 g/L to about 30 g/L, or from about 4 g/L to about 20 g/L, or from about 5 g to about 10 g/L, or from about 0.05 g/L to about 1 g/L, based on the total volume of the oral composition.

In some embodiments of the present method, the oral composition is in a dry or semi dry form. The form of the oral composition may be beverage concentrate, gel, dry powder, tablet, or capsule. In one embodiment, the oral composition is in dry powder that is readily soluble in a drinkable medium. In some embodiments, the method include preparing a drinkable solution containing the dry powder by dissolving the dry powder in a drinkable medium comprising water, and administering the drinkable solution orally. In some embodiments, the concentration of the amino acid formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, based on the total volume of the drinkable solution.

In other embodiments, the method include ingesting/consuming the oral composition and a drinkable medium comprising water. Ingestion of the oral composition and a drinkable medium can be concurrently, simultaneously, separately, or successively. In some embodiments, the ratio of the oral composition to the drinkable medium is such that the content of the amino acid formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, relative to the total volume of the drinkable medium.

In one particular embodiment of the present method, the amino acid formulation consists essentially of glycine, or a glycine-containing dipeptide, or a carbohydrate-glycine complex, or any combinations thereof.

In some embodiments of the present method, the plasma volume of the human is increased by at least about 2.5%, or at least about 3%, or at least about 4% on or before 10 minutes after administration of the oral composition.

In some embodiments, the plasma volume of the human is increased by at least about 4.5%, or at least about 5%, or at least about 5.5%, or at least about 6% on or before 15 minutes after administration of the oral composition.

In some embodiments, the plasma volume of the human is increased by at least about 5%, or at least about 5.5%, or at least about 6%, or at least about 6.5%, or at least about 7% on or before 30 minutes after administration of the oral composition.

In some embodiments, the plasma volume of the human is increased by at least about 3%, or at least about 3.5%, or at least about 4%, or at least about 5%, or at least about 6% on or before 45 minutes after administration of the oral composition.

In some embodiments, the plasma volume of the human is increased by at least about 3%, or at least about 3.5%, or at least about 4%, or at least about 5%, or at least about 6% on or before 60 minutes after administration of the oral composition.

In some embodiments, the plasma volume of the human measured at 30 minutes after administration of the oral composition remains substantially unchanged thereafter 15 for at least about 15 minutes, or at least about 30 minutes, or at least about 1 hour.

In some embodiments, the plasma volume of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.

In some embodiments of the present method, the plasma osmolality of the human is maintained in a range from about 270 to about 330 mOsm/kg, or from about 280 to about 320 mOsm/kg, or from about 290 to about 310 mOsm/kg, or from about 290 to about 300 mOsm/kg, in at least 60 minutes after administration of the oral 25 composition.

In some embodiments, the change of the plasma osmolality of the human is no greater than 3 mOsm/kg in at least 60 minutes after administration of the oral composition.

### Definition and Interpretation of Selected Terms

As used herein, “weight percent,” “wt %, “percent by weight,” “% by weight,” and variations thereof refer to the concentration of a substance as the weight of that substance divided by the total weight of the composition and multiplied by 100. It is understood that, as used here, “percent,” “%,” and the like are intended to be synonymous with “weight percent,” “wt %,” etc.

As used herein, “g” represents gram; “kg” represents kilogram or 1000 grams; “L” represents liter, “mg” represents “milligram (10⁻³ gram);” “mL” or “cc” represents milliliter (10⁻³ liter). The units “g/100 g,” “g/100 mL,” or “g/L” are units of concentration or content of a component in a composition. One “mg/L” equals to one ppm (part per million). “Da” refers to Dalton, which is the unit for molecular weight; One Da equals to one g/mol. The unit of temperature used herein is degree Celsius (° C.

The term “about” is used in conjunction with numeric values to include normal variations in measurements as expected by persons skilled in the art, and is understood to have the same meaning as “approximately” and to cover a typical margin of error, such as ±15%, ±10%, ±5%, ±1%, ±0.5%, or even ±0.1% of the stated value. The term “about” also encompasses amounts that differ due to different equilibrium conditions for a composition resulting from a particular initial composition. Whether or not modified by the term “about,” the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes having two or more compounds that are either the same or different from each other. It should also be noted that the term “or” is generally employed in its sense including “and/or” unless the content clearly dictates otherwise.

As used herein, “and/or” refers to and encompasses any and all possible combinations of one or more of the associated listed items, as well as the lack of combinations when interpreted in the alternative (“or”).

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The term “substantially free” may refer to any component that the composition of the disclosure lacks or mostly lacks. When referring to “substantially free” it is intended that the component is not intentionally added to compositions of the disclosure. Use of the term “substantially free” of a component allows for trace amounts of that component to be included in compositions of the disclosure because they are present in another component. However, it is recognized that only trace or de minimus amounts of a component will be allowed when the composition is said to be “substantially free” of that component. Moreover, if a composition is said to be “substantially free” of a component, if the component is present in trace or de minimus amounts it is understood that it will not affect the effectiveness of the composition. It is understood that if an ingredient is not expressly included herein or its possible inclusion is not stated herein, the disclosure composition may be substantially free of that ingredient. Likewise, the express inclusion of an ingredient allows for its express exclusion thereby allowing a composition to be substantially free of that expressly stated ingredient.

The term “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 1%, 5%, 10%, 15%, 25%, 50%, 75%, 100%, 150%, 200%, 300%, 400%, 500/0 or more as compared to a control.

As used herein, the terms “reduce,” “reduced,” “reducing,” “reduction,” “diminish,” and “decrease” (and grammatical variations thereof), describe, for example, a decrease of at least about 1%, 5%, 10%, 15%, 20%, 25%, 35%, 50%, 75%, 80%, 85%, 90%, 95%, 97%, 98%, 99%, or 100% Y as compared to a control. In particular embodiments, the reduction can result in no or essentially no (i.e., an insignificant amount, e.g., less than about 10% or even 5% or even 1%) detectable activity or amount.

“Osmolality” is defined as the number of dissolved particles in a unit volume of water solution. Osmolarity is defined as the number of dissolved particles in a unit weight of water solution. As a practical matter, osmolality and osmolarity have numerical values which are very close in the ranges involved in the present disclosure and therefore are used interchangeably. A solution that has 1/1000 of an osmol dissolved per kilogram has a concentration of 1 milliosmols (“mOs”) per kilogram. An osmol is the number of particles in 1 gram molecular weight of undissociated solute. Tonicity is a measure of the osmotic pressure of a solution relative to the osmotic pressure of the blood fluids. It is to be understood that the osmotic pressure of the body varies somewhat from one person to the other person. A hypotonic solution is a solution of lower osmotic pressure or tonicity than that of blood. The osmolality of a hypotonic solution is usually in the range of about 80-250 mOs/kg. An isotonic solution has the same tonicity as blood. Here, the osmolality usually ranges from about 280 to about 310 mOs/kg. A hypertonic solution is a solution of greater tonicity than blood. It normally has an osmolality range of about 310-440 mOs/kg. Water has the osmolality of about 10-20 mOs/kg.

The term “beverage” as used herein means any drinkable liquid or semi-liquid, including for example water, flavored water, soft drinks, fruit drinks, tea-based drinks, juice-based drinks, gel drinks, carbonated or non-carbonated drinks, and alcoholic or non-alcoholic drinks. In some embodiments, a beverage powder may first be mixed with any drinkable liquid or semi-liquid to obtain a beverage.

As used herein dehydration is defined as a condition that occurs when the body loses too much water and other fluids that it needs to work normally. Dehydration is usually caused by severe diarrhea and vomiting, but it may also be caused by not drinking enough water or other fluids, sweating too much, fever, urinating too much, taking certain medicines, or physical exertion. Rehydration is the replenishment of water and electrolytes lost through dehydration. Rapid rehydration (or rapid hydration) is the replenishment of water and electrolytes within 30 minutes.

As used herein, “amino acid” refers to an organic compound or unit that contains amino (—NH₂) and carboxyl (—COOH) functional groups. The “amino acid” of the present disclosure broadly encompasses any compound having at least one amino acid unit. The “amino acid formulation” used herein refers to a molecule, a compound, a complex, an oligomer, a polymer, a mixture, or a composition having at least one amino acid unit physically (through non-covenant bonding) or chemically (though covalent, hydrogen, or coordinate bonding) incorporated in the amino acid formulation. Non-limiting examples of amino acid, amino acid compound, and amino acid formulation used herein include aspartic acid, alanine, glycine, glutamic acid, praline, threonine, theanine, cysteine, cystine, alanine, valine, tyrosine, leucine, arabinose, trans-4-hydroxyproline, isoleucine, asparagine, serine, lysine, histidine, omithine, methionine, carnitine, aminobutyric acid (α-, β-, and/or γ-isomers), glutamine, hydroxyproline, taurine, norvaline, sarcosine, and their salt forms such as sodium or potassium salts or acid salts. The amino acid also may be in the D- or L-configuration and in the mono-, di-, or tri-form of the same or different amino acids Additionally, the amino acids may be α-, β-, and/or γ-isomers if appropriate. Combinations of the foregoing amino acids and their corresponding salts (e.g., sodium, potassium, calcium, magnesium salts or other alkali or alkaline earth metal salts thereof, or acid salts) also are suitable additives in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine As used herein, modified amino acids encompass both modified and unmodified amino acids. As used herein, amino acids also encompass both peptides, oligopeptides, and polypeptides (e.g., dipeptides, tripeptides, tetrapeptides, and pentapeptides) such as glutathione and L-alanyl-L-glutamine. Suitable polyamino acid include poly-L-aspartic acid, poly-L-lysine (e.g., poly-L-α-lysine or poly-L-ε-lysine), poly-L-ornithine (e.g., poly-L-α-ornithine or poly-L-ε-ornithine), poly-L-alanine, other polymeric forms of amino acids, and salt forms thereof (e.g., calcium, potassium, sodium, or magnesium salts such as L-glutamic acid mono sodium salt). The poly-amino acid additives also may be in the D- or L-configuration Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts or other alkali or alkaline earth metal salts thereof or acid salts) also are suitable additives in some embodiments. The poly-amino acids described herein also may comprise co-polymers of different amino acids. The poly-amino acids described herein also may comprise co-polymers of different amino acids. The poly-amino acids may be natural or synthetic.

The poly-amino acids also may be modified, such that at least one atom has been added, removed, substituted, or combinations thereof (e.g., N-alkyl polyamino acid or N-acyl poly-amino acid). As used herein, poly-amino acids encompass both modified and unmodified poly-amino acids. For example, modified poly-amino acids include, but are not limited to, poly-amino acids of various molecular weights (MW), such as poly-L-α-glycine with a MW of about 100, about 200, about 300, about 500, about 1,000, about 1,500, about 6,000, about 25,200, about 63,000, about 83,000, or about 300,000 in Dalton (Da). The amino acid formulation provided herein also include an amino acid derivative, a peptide derivate, a peptide hydrolysate or a peptide residue thereof.

### BRIEF DESCRIPTION OF THE DRAWINGS

FIG. 1 shows the plasma volume changes over time after for various compositions according to Example 1.

FIG. 2 shows the plasma osmolality changes over time for various compositions according to Example 1.

### DETAILED DESCRIPTION

### Amino Acid Formulation

In one aspect, the present disclosure relates to a composition for oral consumption comprising an amino acid formulation. Amino acids or formulations containing amino acids have been found to increase water absorption in the human intestine in intestinal perfusion tests, which are designed to understand the intestinal water absorption speed. In this experimental approach, amino acid solutions are directly perfused into the intestine and fluid absorption is measured in a specific segment of the intestine. This method provides valuable insights but does not account for gastric emptying rate and does not provide a direct measurement of the rate of fluid appearance in blood. Six amino acids as shown in Table 1 were previously reported to have effect on electrolyte and water absorption. For example, Hellier et al. reported a perfusion technique used to quantitate the effect of the amino acids glycine and alanine and the dipeptides glycyl-glycine (Gly-Gly) and glycyl-L-alanine (Gly-Ala) on sodium and water absorption from the human jejunum. A significant stimulation occurred in the presence of both free amino acids and dipeptides. In the case of glycine and alanine, it was found that sodium and water absorption increased linearly with increasing concentration of the amino acid and saturation was not demonstrated over the range of concentrations.

TABLE 1
Amino acid formulations of prior disclosures.

| | Dosage | |
|---|---|---|
| Amino Acids | (Intestinal perfusion during rest) | Reference |
| Arginine | 10, 20 mM | Hellier 1973 |
| | | Hegary, 1982 |
| Leucine | 20 mM | Adibi, 1970 |
| Glycine | 10, 20, 50 mM | Hellier, 1973 |
| Alanine | 10, 20, 50 mM | Hellier, 1973 |
| Glutamine | 90 mM | Coeffier, 2005 |
| Glycine + | 10 mM for each | Hellier, 1973 |
| Alanine | 10, 20, 40 mM for each | Silk, 1975 |
| | | (Glycine + L-Alanine) |
| Glycyl-Alanine | 10 mM | Hellier, 1973 |
| (Dipeptide) | 10, 20, 40 mM | Silk, 1975 |
| | | (Glycyl-L-Alanine) |
| Glycyl-glycine | 10 mM | Hellier, 1973 |
| (Dipeptide) | | |

It is noted that, however, no persuasive data has been provided so far to support that amino acids have positive effect on retention of body fluid. Several examples of compositions containing amino acid that were previously reported are provided in Table 2. For example, Tai et al. investigated several amino acids to compare the rehydration capabilities of an electrolyte-carbohydrate (EC), electrolyte-branched chain amino acid (EA), and flavored water (FW) beverages. However, no significant differences (p >0.05) existed between beverages for urine volume, drink volume, or fluid retention for any time-point. Sollanek et al. and Cheuvront et al. each reported a study on rehydration effect of beverages containing ENTERADE® compared with control beverages without amino acid ENTERADE® is a commercial food product containing five selected amino acids: threonine, aspartic acid, tyrosine, serine, and valine. However, no direct and clear rehydration effect was provided with respect to the contributing factor of each amino acid, and the beverages compared in the experiments significantly differed from each other with respect to the composition.

TABLE 2
Rehydration compositions of prior disclosures.

| | | AA Dosage | | | | |
|---|---|---|---|---|---|---|
| | | (Rehydration | | | | |
| Reference | Amino Acids | after exercise) | Na+ | | Carbohydrate | |
| Tai 2014 | AMINOLTM | 7.8 g/L (BCAA) | 4.5 | mM/L | 2.59 | g/L |
| | (BCAA) | | | | | |
| | MusclePharm | | | | | |
| | GATORADE ® | — | 17 | mM/L | 67.7 | g/L |
| | Flavored water | — | 0 | | 0 | |
| Sollanek | ENTERADE ® | 5 g/L (calculation | 55 | mM/L | 0 | |
| 2018 | | based on label) | | | | |
| | PEDIALYTE | | 44 | mM/L | 25 | g/L |
| | GATORADE ® | — | 20 | mM/L | 61 | g/L |
| | Water | | | | | |
| Cheuvront | ENTERADE ® | 5 g/L (calculation | 67 | mM/L | 0 | |
| 2018 | | based on label) | | | | |
| | GATORADE ® | | 21 | mM/L | 58 | g/L |

Further, it has been reported that protein consumption alone, or protein consumption with a carbohydrate beverage can induce rapid de nova hepatic protein synthesis which can elevate plasma volume after exercise-heat stress from dehydration. In addition, heat stress and dehydration—both alone—can induce increased total circulating protein—likely partially due to de novo hepatic protein synthesis. For example, elevation of total circulating protein and plasma volume was found after 3-4 hr or longer period rehydration. (Okazaki et al., 2002).

The present disclosure is based, at least in part, on the findings that amino acid formulation added in a beverage could body fluid volume improvement after exercise and rehydration. A better sustained and replenished body fluid— after rehydration will provide possible thermoregulatory and performance advantages during subsequent exercise-heat stress.

The present disclosure advantageously provides compositions that are effective in improving the rehydration and retention of hydration. The amino acid formulation according to the present disclosure may be an amino acid, or an amino acid compound, or an amino acid-containing composition, or mixtures thereof.

As described herein, a randomized crossover design study was conducted with healthy male participants using modified Beverage Hydration Index (B11) protocol (Maughan et al. 2016). Subjects visited the lab for five times to test three different test beverages. The test beverage compositions were 1. Aquarius (Aqua) (4% carbohydrate+18 mmol/L sodium), 2. Low carbohydrate (Low CHO) (3% carbohydrate+18 mmol/L sodium+Amino Acid), 3. PowerAde Zero (Zero CHO) (0% carbohydrate+18 mmol/L sodium+Amino Acid)]. The study also included water and PowerAde (PWDE) as controls. Faster absorption was measured by using deuterium oxide (D₂O) technique (Jeukendrup et al. 2009). Participants ingested 0.5 L of beverages on each trial, and the appearance of D₂O in blood stream was analyzed for 60 mins. Longer lasting hydration effect was measured with net fluid balance (NFB), calculated from estimated sweat loss, volume of fluid ingested, and urine output, overtime and with BHI at each time point up through 2 hours post one-liter ingestion. As shown in Table 3, low CHO appeared in the blood stream significantly faster than PWDE at 10, 15, 20 and 25 min time point (p<0.05) representing faster hydration. PWDE showed greater NFB and BHI than Water at 30, 60, 90, and 120 min time point (P<0.05) representing longer lasting hydration. However, there were no significant differences found among Low CHO, Aqua, and Water (p>0.05) representing no detrimental effect of longer lasting hydration. It was surprising that low CHO not only appeared to hydrate faster than the traditional PWDE, but also did not show any detrimental effect compared to the traditional PWDE sample.

Referring still to Table 3, where low CHO appeared in the blood stream significantly faster than PWDE at 10, 15, 20 and 25 mm time point (p<0.05) representing faster hydration. PWDE showed greater NFB and BHT than Water at 30, 60, 90, and 120 min time point (P<0.05) representing longer lasting hydration. However, there were no significant differences found among low CHO, Aqua, and Water (p>0.05) representing no detrimental effect of longer lasting hydration. It should be appreciated form the data above that low CHO not only appeared faster than the traditional PWDE, but also did not show any detrimental effect compared to the traditional PWDE. A double-blind, placebo-controlled study was also conducted with 16 healthy participants (10 male) in a randomized, cross-over design using dehydration/rehydration protocol (Shirreffs 1998). Subjects visited the lab for four times to test two test beverages, the first test beverage was designated as ORS (2% carbohydrate+42.6 mmol/L sodium), the second test beverage was a protein added beverage (PRO) (4% carbohydrate+18 mmol/L sodium+20 g/L whey protein). Both test beverages where tested as rehydration solutions after dehydration induced with exercise in the heat, by having water and PowerAde (PWDE) as controls. Faster absorption was measured by using deuterium oxide (D₂O) technique. Participants ingested D₂O with rehydration beverages to replenish 150% of body weight loss, and the appearance of D₂O in blood stream was analyzed for 60 mins. Longer lasting hydration effect was measured with net fluid balance (NFB) calculated from estimated sweat loss, volume of fluid ingested, and urine output, over three hours post rehydration.

As shown in Table 4, ORS appeared in the blood stream significantly faster than Water and PWDE at 15, 20, 25, 30, 45, and 60 min time point (p<0.05) representing faster hydration. For longer lasting hydration, both ORS and PRO showed greater NFB than Water at 120 and 180 min time point (p<0.05). The results indicated that ORS not only appeared faster than the traditional PWDE and water, but also lasted longer compared to water. PRO also showed the longer lasting hydration effect than water.

In some embodiments, the amino acid formulation comprises an amino acid. Non-limiting examples of amino acid include leucine, isoleucine, valine, histidine, lysine, methionine, phenylalanine, threonine, and tryptophan, or any combination thereof. These amino acids can be categorized as essential amino acids. Other non-limiting examples of amino acid include alanine, arginine, asparagine, aspartic acid, cysteine, glutamic acid, glutamine, glycine, proline, serine, and tyrosine, or any combination thereof. These amino acids can be categorized as non-essential amino acids.

In at least one example embodiment, the amino acid formulation will comprise at least one essential amino acid and at least one non-essential amino acid. In other embodiments, the amino acid formulation consists essentially of glycine and is free or substantially free from other amino acid. In one embodiment, the amino acid formulation consists essentially of alanine and is free or substantially free from other amino acid. In one embodiment, the amino acid formulation consists essentially of glycine and alanine in a ratio from about 100:1 (w/w) to about 1:100 (w/w), and is free or substantially free from other amino acid. It was surprisingly found that the present compositions comprising glycine had excellent performance in rapid hydration, prolonged retention of plasma volume, and prolonged retention of plasma osmolality.

In some embodiments, the amino acid formulation comprises a dipeptide. Non-limiting examples of dipeptide includes glycine-containing dipeptide, lysine-containing dipeptide, alanine-containing dipeptide, alanine-containing dipeptide, glutamine-containing dipeptide More examples of dipeptide include L-alanyl-L-glutamine (L-Ala-L-Gln), glycyl-glycine (Gly-Gly), L-glutamyl-L-alanine (Glu-Ala), Gly-Ala, Ala-Gly, Glu-Gly, Gly-Glu, Glu-Ala, Ala-Glu, commercial dipeptides such as aspartame, carnosine, acetylcarnosine, Val-Tyr, or any stereoisomers thereof, or any salt or derivatives thereof, or any combination thereof.

In one particular embodiment, the amino acid formulation consists essentially of a compound selected from the group consisting of Gly-Gly, Ala-Ala, Gly-Ala, Ala-Gly, or salts and derivatives thereof, or any combinations thereof. In one embodiment, the amino acid formulation consists essentially of Gly-Gly.

In some embodiments, the amino acid formulation comprises a carbohydrate-amino acid complex. The carbohydrate-amino acid complex used herein refers to a compound having both a carbohydrate unit and an amino acid unite that are integrated in the compound through covalent bonding. The carbohydrate unit can be a single sugar molecule or oligosaccharides or polysaccharides. Non-limiting examples of carbohydrate include sucrose, glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, fucose, rhamnose, arabinose, turanose, sialose. Non-limiting examples of carbohydrate-amino acid complex include glutamine-glucose, alanine-glucose, glycine-glucose, alanine-glucose, lysine-glucose, or combinations thereof.

In one particular embodiment, the amino acid formulation consists essentially of a compound selected from the group consisting of glutamine-glucose, alanine-glucose, glycine-glucose, alanine-glucose, or salts and derivatives thereof, or any combinations thereof.

In some embodiments, the amino acid formulation comprises a compound selected from the group consisting of an amino acid, a dipeptide, a carbohydrate-amino acid complex, or any combination thereof. In one particular embodiment, the amino acid formulation consists essentially of an amino acid and a dipeptide, wherein the amino acid is glycine or alanine or both, and wherein the dipeptide is Gly-Gly, Ala-Ala, Gly-Ala, Ala-Gly, or any combinations thereof.

The concentration of the amino acid formulation in the composition can be from about 1 g/L to about 50 g/L, or from about 2 g/L to about 30 g/L, or from about 3 g/L to about 30 g/L, or from about 4 g/L to about 20 g/L, or from about 5 g/L, to about 10 g/L, or from about 0.05 g/L, to about 1 g/L, based on the total volume of the composition.

In one particular embodiment, the present composition comprises glycine as the only amino acid ingredient, wherein the glycine concentration is from about 4 g/L to about 20 g/L, based on the total volume of the composition.

In some embodiments, the present composition comprises a branched-chain amino acid (BCAA). A branched-chain amino acid is an amino acid having an aliphatic side-chain with a branch (a central carbon atom bound to three or more carbon atoms). There are three proteinogenic BCAA: leucine, isoleucine, and valine Non-proteinogenic BCAAs include 2-aminoisobutyric acid. In a more particular embodiment, a beverage comprises leucine, isoleucine and valine. The BCAA can be in the D- or L-configuration.

The present composition may optionally contain a total BCAA in a concentration from about 50 mg/L (ppm) to about 5,000 mg/L, such as, for example, from about 1,000 mg/L to about 5,000 mg/L, from about 2,000 mg/L to about 5,000 mg/L, from about 3,000 mg/L to about 5,000 mg/L, from about 4,000 mg/L to about 5,000 mg/L, from about 1,000 mg/L to about 4,000 mg/L, from about 2,000 mg/L to about 4,000 mg/L, from about 3,000 mg/L to about 4,000 mg/L, from about 1,000 mg/L to about 3,000 mg/L, from about 2,000 mg/L to about 3,000 mg/L or from about 1,000 mg/L to about 2,000 mg/L. In a particular embodiment, a beverage of the present disclosure contains about 2,000 mg/L to about 3,000 mg/L BCAA.

In other embodiments, the present composition is free or substantially free from a branched-chain amino acid (BCAA).

### Additional Ingredients

The present oral composition can contain additional typical beverage ingredients, e.g. at least one electrolyte and/or at least one sweetener and/or at least one functional ingredient and/or at least one additive.

### Electrolyte

The present composition may contain at least one electrolyte. Non limiting examples of electrolytes include sodium, potassium, calcium, magnesium, chloride, phosphate, bicarbonate, and combinations thereof. The electrolytes and ionic components for the present disclosure are usually, but not necessarily, obtainable from their corresponding water-soluble and non-toxic salts Unless otherwise defined, the amount of electrolytes or ionic components in the composition is based on those present in the final ingestible form. The electrolyte concentration is of the ion only and not the salt.

The present composition preferably contains a total electrolyte concentration of at least about 200 mg/L, at least about 300 mg/L, at least about 400 mg/L, at least about 500 mg/L, at least about 600 mg/L, at least about 700 mg/L or at least about 800 mg/L.

In a particular embodiment, the present composition contains an electrolyte concentration from about 400 mg/L to about 1,000 mg/L, from about 400 mg/L to about 900 mg/L, from about 400 mg/L to about 800 mg/L, from about 400 mg/L to about 700 mg/L, from about 400 mg/L to about 600 mg/L, from about 400 mg/L to about 500 mg/L, from about 500 mg/L, to about 1,000 mg/L.

The potassium ion component can be provided by any salt including the chloride, carbonate, sulfate, acetate, bicarbonate, citrate, phosphate, hydrogen phosphate, tartrate, sorbate or a combination thereof. The potassium ions are preferably present in the composition of the present disclosure in an amount of at least 0.0025% to about 0.08% by weight, from about 0.0075% to about 0.06% or from about 0.0075% to about 0.015% by weight.

The present composition can contain from about 5 mg/L to about 1,000 mg/L potassium, more preferably from about 50 mg/L to about 300 mg/L, such as, for example, from about 100 mg/L to about 300 mg/L, from about 200 mg/L to about 300 mg/L, from about 50 mg/L to about 200 mg/L, from about 100 mg/L to about 200 mg/L or from about 100 mg/L to about 200 mg/L.

The sodium ion component can be provided by any salt such as the chloride, carbonate, sulfate, acetate, bicarbonate, citrate, phosphate, hydrogen phosphate, tartrate, sorbate, lactate or a combination thereof. The sodium ions are preferably present in the present composition in an amount of at least about 0.005% to about 0.1% by weight, from about 0.0075% to about 0.075% or about 0.015% to about 0.05% by weight.

The present composition can contain from about 5 mg/L to about 1,000 mg/L sodium, more preferably from about 300 mg/L to about 800 mg/L sodium, such as, for example, from about 300 mg/L to about 700 mg/L, from about 300 mg/L to about 600 mg/L, from about 300 mg/L to about 500 mg/L, from about 300 mg/L to about 400 mg/L, from about 400 mg/L to about 800 mg/L, from about 400 mg/L to about 700 mg/L, from about 400 mg/L to about 600 mg/L from about 400 mg/L to about 500 mg/L, from about 500 mg/L to about 800 mg/L, from about 500 mg/L to about 700 mg/L, from about 500 mg/L to about 600 mg/L, from about 600 mg/L to about 800 mg/L, from about 600 mg/L to about 700 mg/L and from about 700 mg/L to about 800 mg/L. In a particular embodiment, the present composition contains from about 600 mg/L to about 700 mg/L sodium.

The calcium ion component can be provided by any salt such as the chloride, carbonate, sulfate, acetate, bicarbonate, citrate, phosphate, hydrogen phosphate, tartrate, sorbate or a combination thereof. The calcium ions are preferably present in the present composition in an amount of at least about 0.0005% to about 0.010% by weight.

The present composition can contain from about 5 mg/L to about 1,000 mg/L calcium, more preferably from about 1 mg/IL to about 50 mg/L, such as, for example, from about 5 mg/L to about 10 mg/L. The magnesium ion component can be provided by any salt such as the chloride, carbonate, sulfate, acetate, bicarbonate, citrate, phosphate, hydrogen phosphate, tartrate, sorbate or a combination thereof. The magnesium ions are preferably present in the present composition in an amount of at least about 0.0005% to about 0.010% by weight.

The present composition can contain from about 5 mg/L to about 1,000 mg/L magnesium, more preferably from about 1 mg/L to about 50 mg/L, such as, for example, from about 5 mg/L to about 20 mg/L.

The present composition can contain chloride ion from about 0.005% to about 0.20% by weight, from about 0.01% to about 0.15% or from about 0.02% to about 0.075%. The chloride ion component can be provided by a salt such as sodium chloride, potassium chloride or a combination thereof.

In a particular embodiment, a beverage of the present disclosure contains at least one electrolyte selected from the group consisting of sodium, potassium, magnesium, calcium and combinations thereof. In another particular embodiment, a beverage of the present disclosure contains at least one electrolyte selected from the group consisting of sodium, potassium, magnesium, calcium and combinations thereof, wherein the amount of each electrolyte is as provided above.

### Sweetener

The present composition may optionally include a sweetener. The sweetener can be an artificial or synthetic sweetener, a natural sweetener, a natural high potency sweetener. As used herein, the phrase “natural high potency sweetener” (NHPS) refers to any sweetener found naturally in nature and characteristically has a sweetness potency greater than sucrose, fructose, or glucose, yet has less calories. The natural high potency sweetener can be provided as a pure compound or, alternatively, as part of an extract. As used herein, the phrase “synthetic sweetener” refers to any composition which is not found naturally in nature and characteristically has a sweetness potency greater than sucrose, fructose, or glucose, yet has less calories.

Non-limiting examples of NHPSs includes stevia and steviol glycosides, such as rebaudioside M, rebaudioside D, rebaudioside A, rebaudioside N, rebaudioside O, rebaudioside E, steviolmonoside, steviolbioside, rubusoside, dulcoside B, dulcoside A, rebaudioside B, rebaudioside G, stevioside, rebaudioside C, rebaudioside F, rebaudioside 1, rebaudioside H, rebaudioside L, rebaudioside K, rebaudioside J, rebaudioside M2, rebaudioside D2, rebaudioside S, rebaudioside T, rebaudioside U, rebaudioside V, rebaudioside W, rebaudioside Z1, rebaudioside Z2, rebaudioside IX, enzymatically glucosylated steviol glycosides and combinations thereof. Examples of high purity steviol glycosides and methods of making the same are provided in U.S. Pat. App. No. 2021/0107933, which is hereby incorporated in its entirety.

In certain embodiments, a steviol glycoside blend comprises at least about 5% steviol glycoside by weight, such as, for example, at least about 10%, at least about 20%, at least about 30%, at least about 40%, at least about 50%, at least about 60%, at least about 700%, at least about 80%, at least about 90%, at least about 95% or at least about 97%. In exemplary embodiments, the steviol glycoside blend comprises at least about 50% steviol glycoside by weight, such as, for example, from about 50% to about 90%, from about 50% to about 80%, from about 50% to about 70%, from about 50% to about 60%, from about 60% to about 90%, from about 60% to about 80%, from about 60% to about 70%, from about 70%, to about 90%6, from about 70% to about 80% and from about 80% to about 90%.

Another exemplary NHPS is Luo Han Guo and the related mogroside compounds, such as grosmogroside I, mogroside IA, mogroside IE, 11-oxomogroside IA, mogroside II mogroside II A, mogroside II B, mogroside II E, 7-oxomogroside II E, mogroside III, Mogroside HIE, 11-oxomogroside HIE, 11-deoxymogroside III, mogroside IV, Mogroside iVA 11-oxomogroside IV, 11-oxomogroside IVA, mogroside V, isonogroside V, 11-deoxymogroside V, 7-oxomogroside V, 11-oxomogroside V, isomogroside V, mogroside VI, mogrol, 11-oxomogrol, siamenoside I, isomers of siamenoside I (e.g. those disclosed in US Pat. App. No. 20170119032; incorporated by reference in its entirety), (3β, 9β, 10α, 11α, 24R)-3-[(4-O-β-D-glucospyranosyl-6-O-β-D-glucopyranosyl]-25-hydroxyl-9-methyl-19-norlanost-5-en-24-yl-[2-O-β-D-glucopyranosyl-6-O-β-D-glucopyranosyl]-β-D-glucopyranoside); (3β, 9β, 10α, 11α, 24R)-[(2-O-β-D-glucopyranosyl-6-O-β-D-glucopyranosyl-β-D-glucopyranosyl)oxy]-25-hydroxy-9-methyl-19-norlanost-5-en-24-yl-[2-O-β-D-glucopyranosyl-6-O-β-D-glucopyranosyl]-β-D-glucopyranoside); and (3β, 9β, 10α, 11α, 24R)-[(2-O-β-D-glucopyranosyl-6-O-β-D-glucopyranosyl-β-D-glucopyranosyl)oxy]-25-hydroxy-9-methyl-19-norlanost-5-en-24-yl-[2-O-β-D-glucopyranosyl-6-O-β-D-glucopyranosyl]-β-D-glucopyranoside).

In certain embodiments, a mogroside blend comprises at least about 5% of the mogroside by weight, such as, for example, at least about 10%, at least about 20%, at least about 30%, at least about 40%, at least about 50%, at least about 60%, at least about 70%, at least about 80%, at least about 90%, at least about 95% or at least about 97%.

Other exemplary NHPSs include monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I periandrin I, abrusoside A, and cyclocarioside I.

In one embodiment, the sweetener is a carbohydrate sweetener. Suitable carbohydrate sweeteners include, but not limited to, the group consisting of sucrose, glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, fucose, rhamnose, arabinose, turanose, sialose and combinations thereof.

In certain embodiments, the present composition is free or substantially free from a carbohydrate sweetener.

Other suitable sweeteners include siamenoside, monatin and its salts (monatin SS, RR, RS, SR), curculin, mogrosides, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside 1, periandrin I, abrusoside A, steviolbioside and cyclocarioside 1, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof: neotame, advantame, glucosylated steviol glycosides (GSGs) and combinations thereof.

In one embodiment, the sweetener is a caloric sweetener or mixture of caloric sweeteners. In another embodiment, the caloric sweetener is selected from sucrose, fructose, glucose, high fructose com/starch syrup, a beet sugar, a cane sugar, and combinations thereof.

In certain embodiments, the present composition is free or substantially free from a caloric sweetener.

In another embodiment, the sweetener is a rare sugar selected from allulose, gulose, kojibiose, sorbose, lyxose, ribulose, xylose, xylulose, D-allose, L-ribose, D-tagatose, L-glucose, L-fucose, L-arabinose, turanose and combinations thereof.

The amount of sweetener in the present composition depends on the identity of the sweetener and the desired level of sweetness. In preferred embodiments, the sweetener is present in a sweetening amount, i.e. a concentration that is detectably sweet.

As would be understood by a person of skill in the art, high potency sweeteners are more potent and therefore lower concentrations are required to achieve a particular sucrose equivalence (SE). The sweetness of a non-sucrose sweetener can be measured against a sucrose reference by determining the non-sucrose sweetener's sucrose equivalence (SE). Typically, taste panelists are trained to detect sweetness of reference sucrose solutions containing between 1-15% sucrose (w/v). Other non-sucrose sweeteners are then tasted at a series of dilutions to determine the concentration of the non-sucrose sweetener that is as sweet as a given percent sucrose reference. For example, if a 1% solution of a non-sucrose sweetener is as sweet as a 10% sucrose solution, then the sweetener is said to be 10 times as potent as sucrose, and has 10% sucrose equivalence.

In one embodiment, the sweetener or sweeteners provides the present composition with a sucrose equivalence of about 1% (w/v), such as, for example, about 2%, about 3%, about 4%, about 5%, about 6%, about 7%, about 8%, about 9%, about 10%, about 11%, about 12%, about 13%, about 14% or any range between these values.

In another embodiment, the present composition has a SE from about 2% to about 14%, such as, for example, from about 2% to about 10%, from about 2% to about 5%, from about 5% to about 15%, from about 5% to about 10% or from about 10% to about 15%.

The amount of sucrose, and thus another measure of sweetness, in a reference solution may be described in degrees Brix (°Bx). One degree Brix is I gram of sucrose in 100 grams of solution and represents the strength of the solution as percentage by weight (% w/w) (strictly speaking, by mass). In embodiments where the present composition is sweetened with sucrose, the beverage can be about 1 degree Brix, about 2 degrees Brix, about 3 degrees Brix, about 4 degrees Brix, about 5 degrees Brix, about 6 degrees Brix, about 7 degrees Brix, about 8 degrees Brix, about 9 degrees Brix, about degrees Brix, about 11 degrees Brix, about 12 degrees Brix, about 13 degrees Brix, about 14 degrees Brix or any range between these values.

### Functional Components/Ingredients

The present composition may optionally include a functional ingredient. Exemplary functional ingredients include, but are not limited to, saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

In certain embodiments, the functional ingredient is at least one saponin. As used herein, the at least one saponin may comprise a single saponin or a plurality of saponins as a functional ingredient for the composition provided herein. Saponins are glycosidic natural plant products comprising an aglycone ring structure and one or more sugar moieties. Non-limiting examples of specific saponins for use in particular embodiments of the disclosure include group A acetyl saponin, group B acetyl saponin, and group E acetyl saponin. Several common sources of saponins include soybeans, which have approximately 5% saponin content by dry weight, soapwort plants Saponaria), the root of which was used historically as soap, as well as alfalfa, aloe, asparagus, grapes, chickpeas, yucca, and various other beans and weeds. Saponins may be obtained from these sources by using extraction techniques well known to those of ordinary skill in the art. A description of conventional extraction techniques can be found in U.S. Pat. Appl. No. 2005/0123662.

In certain embodiments, the functional ingredient is at least one antioxidant. As used herein, “antioxidant” refers to any substance which inhibits, suppresses, or reduces oxidative damage to cells and biomolecules.

Examples of suitable antioxidants for embodiments of this disclosure include, but are not limited to, vitamins, vitamin cofactors, minerals, hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polypphenols, nonflavonoid phenolics, isothiocyanates, and combinations thereof. In some embodiments, the antioxidant is vitamin A, vitamin C, vitamin E, ubiquinone, mineral selenium, manganese, melatonin, oc-carotene, b-carotene, lycopene, lutein, zeanthin, crypoxanthin, reservatol, eugenol, quercetin, catechin, gossypol, hesperetin, curcumin, ferulic acid, thymol, hydroxytyrosol, tumeric, thyme, olive oil, lipoic acid, glutathinone, gutamine, oxalic acid, tocopherol-derived compounds, butylated hydroxyanisole (BHA), butylated hydroxytoluene (BHT), ethylenediaminetetraacetic acid (EDTA), tert-butylhydroquinone, acetic acid, pectin, tocotrienol, tocopherol, coenzyme Q10, zeaxanthin, astaxanthin, canthaxantin, saponins, limonoids, kaempfedrol, myricetin, isorhamnetin, proanthocyanidins, quercetin, rutin, luteolin, apigenin, tangeritin, hesperetin, naringenin, erodictyol, flavan-3-ols (e.g., anthocyanidins), gallocatechins, epicatechin and its gallate forms, epigallocatechin and its gallate forms (ECGC) theaflavin and its gallate forms, thearubigins, isoflavone, phytoestrogens, genistein, daidzein, glycitein, anythocyanins, cyaniding, delphinidin, malvidin, pelargonidin, peonidin, petunidin, ellagic acid, gallic acid, salicylic acid, rosmarinic acid, cinnamic acid and its derivatives (e.g., ferulic acid), chlorogenic acid, chicoric acid, gallotannins, ellagitannins, anthoxanthins, betacyanins and other plant pigments, silymarin, citric acid, lignan, antinutrients, bilirubin, uric acid, R-oc-lipoic acid, N-acetylcysteine, emblicanin, apple extract, apple skin extract (applephenon), rooibos extract red, rooibos extract, green, hawthorn berry extract, red raspberry extract, green coffee antioxidant (GCA), aronia extract 20%, grape seed extract (VinOseed), cocoa extract, hops extract, mangosteen extract, mangosteen hull extract, cranberry extract, pomegranate extract, pomegranate hull extract, pomegranate seed extract, hawthorn berry extract, pomella pomegranate extract, cinnamon bark extract, grape skin extract, bilberry extract, pine bark extract, pycnogenol, elderberry extract, mulberry root extract, wolfberny (gogi) extract, blackberry extract, blueberry extract, blueberry leaf extract, raspberry extract, turmeric extract, citrus bioflavonoids, black currant, ginger, acai powder, green coffee bean extract, green tea extract, and phytic acid, or combinations thereof. In alternate embodiments, the antioxidant is a synthetic antioxidant such as butylated hydroxytolune or butylated hydroxyanisole, for example. Other sources of suitable antioxidants for embodiments of this disclosure include, but are not limited to, fruits, vegetables, tea, cocoa, chocolate, spices, herbs, rice, organ meats from livestock, yeast, whole grains, or cereal grains.

Particular antioxidants belong to the class of phytonutrients called polyphenols (also known as “polyphenolics”), which are a group of chemical substances found in plants, characterized by the presence of more than one phenol group per molecule. Suitable polyphenols for embodiments of this disclosure include catechins, proanthocyanidins, procyanidins, anthocyanins, quercerin, rutin, reservatrol, isoflavones, curcumin, punicalagin, ellagitannin, hesperidin, naringin, citrus flavonoids, chlorogenic acid, other similar materials, and combinations thereof.

In one embodiment, the antioxidant is a catechin such as, for example, epigallocatechin gallate (EGCG). In another embodiment, the antioxidant is chosen from proanthocyanidins, procyanidins or combinations thereof. In particular embodiments, the antioxidant is an anthocyanin. In still other embodiments, the antioxidant is chosen from quercetin, rutin or combinations thereof. In one embodiment, the antioxidant is reservatrol. In another embodiment, the antioxidant is an isoflavone. In still another embodiment, the antioxidant is curcumin. In a yet further embodiment, the antioxidant is chosen from punicalagin, ellagitannin or combinations thereof. In a still further embodiment, the antioxidant is chlorogenic acid.

In certain embodiments, the functional ingredient is at least one dietary fiber. Numerous polymeric carbohydrates having significantly different structures in both composition and linkages fall within the definition of dietary fiber. Such compounds are well known to those skilled in the art, non-limiting examples of which include non-starch polysaccharides, lignin, cellulose, methylcellulose, the hemicelluloses, b-glucans, pectins, gums, mucilage, waxes, inulins, oligosaccharides, fiuctooligosaccharides, cyclodextrins, chitins, and combinations thereof. Although dietary fiber generally is derived from plant sources, indigestible animal products such as chitins are also classified as dietary fiber. Chitin is a polysaccharide composed of units of acetylglucosamine joined by b(1-4) linkages, similar to the linkages of cellulose.

In certain embodiments, the functional ingredient is at least one fatty acid. As used herein, “fatty acid” refers to any straight chain monocarboxylic acid and includes saturated fatty acids, unsaturated fatty acids, long chain fatty acids, medium chain fatty acids, short chain fatty acids, fatty acid precursors (including omega-9 fatty acid precursors), and esterified fatty acids. As used herein, “long chain polyunsaturated fatty acid” refers to any polyunsaturated carboxylic acid or organic acid with a long aliphatic tail. As used herein, “omega-3 fatty acid” refers to any polyunsaturated fatty acid having a first double bond as the third carbon-carbon bond from the terminal methyl end of its carbon chain. In particular embodiments, the omega-3 fatty acid may comprise a long chain omega-3 fatty acid. As used herein, “omega-6 fatty acid” any polyunsaturated fatty acid having a first double bond as the sixth carbon-carbon bond from the terminal methyl end of its carbon chain.

Suitable omega-3 fatty acids for use in embodiments of the present disclosure can be derived from algae, fish, animals, plants, or combinations thereof, for example. Examples of suitable omega-3 fatty acids include, but are not limited to, linolenic acid, alpha-linolenic acid, eicosapentaenoic acid, docosahexaenoic acid, stearidonic acid, eicosatetraenoic acid and combinations thereof. In some embodiments, suitable omega-3 fatty acids can be provided in fish oils, (e.g., menhaden oil, tuna oil, salmon oil, bonito oil, and cod oil), microalgae omega-3 oils or combinations thereof. In particular embodiments, suitable omega-3 fatty acids may be derived from commercially available omega-3 fatty acid oils such as Microalgae DHA oil (from Martek, Columbia, MD), OmegaPure (from Omega Protein, Houston, TX), Marinol C-38 (from Lipid Nutrition, Channahon, IL), Bonito oil and MEG-3 (from Ocean Nutrition, Dartmouth, NS), Evogel (from Symrise, Holzminden, Germany), Marine Oil, from tuna or salmon (from Arista Wilton, CT), OmegaSource 2000, Marine Oil, from menhaden and Marine Oil, from cod (from OmegaSource, RTP, NC). Suitable omega-6 fatty acids include, but are not limited to, linoleic acid, gamma-linolenic acid, dihommo-gamma-linolenic acid, arachidonic acid, eicosadienoic acid, docosadienoic acid, adrenic acid, docosapentaenoic acid and combinations thereof.

Suitable esterified fatty acids for embodiments of the present disclosure include, but are not limited to, monoacylgycerols containing omega-3 and/or omega-6 fatty acids, diacylgycerols containing omega-3 and/or omega-6 fatty acids, or triacylgycerols containing omega-3 and/or omega-6 fatty acids and combinations thereof.

In certain embodiments, the functional ingredient is at least one vitamin. Suitable vitamins include, vitamin A, vitamin D, vitamin E, vitamin K, vitamin BI, vitamin B2, vitamin B3, vitamin B5, vitamin B6, vitamin B7, vitamin B9, vitamin B 12, and vitamin C.

Various other compounds have been classified as vitamins by some authorities. These compounds may be termed pseudo-vitamins and include, but are not limited to, compounds such as ubiquinone (coenzyme Q10), pangamic acid, dimethylglycine, taestrile, amygdaline, flavanoids, para-aminobenzoic acid, adenine, adenylic acid, and s-methylmethionine. As used herein, the term vitamin includes pseudo-vitamins. In some embodiments, the vitamin is a fat-soluble vitamin chosen from vitamin A, D, E, K and combinations thereof. In other embodiments, the vitamin is a water-soluble vitamin chosen from vitamin B1, vitamin B2, vitamin B3, vitamin B6, vitamin B12, folic acid, biotin, pantothenic acid, vitamin C and combinations thereof.

In certain embodiments, the functional ingredient is glucosamine, optionally further comprising chondroitin sulfate.

In certain embodiments, the functional ingredient is at least one mineral. Minerals, in accordance with the teachings of this disclosure, comprise inorganic chemical elements required by living organisms. Minerals are comprised of a broad range of compositions (e.g., elements, simple salts, and complex silicates) and also vary broadly in crystalline structure. They may naturally occur in foods and beverages, may be added as a supplement, or may be consumed or administered separately from foods or beverages.

Minerals may be categorized as either bulk minerals, which are required in relatively large amounts, or trace minerals, which are required in relatively small amounts. Bulk minerals generally are required in amounts greater than or equal to about 100 mg per day and trace minerals are those that are required in amounts less than about 100 mg per day. In one embodiment, the mineral is chosen from bulk minerals, trace minerals or combinations thereof. Non-limiting examples of bulk minerals include calcium, chlorine, magnesium, phosphorous, potassium, sodium, and sulfur. Non-limiting examples of trace minerals include chromium, cobalt, copper, fluorine, iron, manganese, molybdenum, selenium, zinc, and iodine. Although iodine generally is classified as a trace mineral, it is required in larger quantities than other trace minerals and often is categorized as a bulk mineral.

In a particular embodiment, the mineral is a trace mineral, believed to be necessary for human nutrition, non-limiting examples of which include bismuth, boron, lithium, nickel, rubidium, silicon, strontium, tellurium, tin, titanium, tungsten, and vanadium.

The minerals embodied herein may be in any form known to those of ordinary skill in the art. For example, in one embodiment, the minerals may be in their ionic form, having either a positive or negative charge. In another embodiment, the minerals may be in their molecular form. For example, sulfur and phosphorous often are found naturally as sulfates, sulfides, and phosphates.

In certain embodiments, the functional ingredient is at least one preservative. In particular embodiments, the preservative is chosen from antimicrobials, antioxidants, antienzymatics or combinations thereof. Non-limiting examples of antimicrobials include sulfites, propionates, benzoates, sorbates, nitrates, nitrites, bacteriocins, salts, sugars, acetic acid, dimethyl dicarbonate (DMDC), ethanol, and ozone. In one embodiment, the preservative is a sulfite. Sulfites include, but are not limited to, sulfur dioxide, sodium bisultite, and potassium hydrogen sulfite. In another embodiment, the preservative is a propionate. Propionates include, but are not limited to, propionic acid, calcium propionate, and sodium propionate. In yet another embodiment, the preservative is a benzoate. Benzoates include, but are not limited to, sodium benzoate and benzoic acid. In still another embodiment, the preservative is a sorbate. Sorbates include, but are not limited to, potassium sorbate, sodium sorbate, calcium sorbate, and sorbic acid. In a still further embodiment, the preservative is a nitrate and/or a nitrite. Nitrates and nitrites include, but are not limited to, sodium nitrate and sodium nitrite. In another embodiment, the at least one preservative is a bacteriocin, such as, for example, nisin. In still another embodiment, the preservative is ethanol. In yet another embodiment, the preservative is ozone. Non-limiting examples of anti-enzymatis suitable for use as preservatives in particular embodiments of the disclosure include ascorbic acid, citric acid, and metal chelating agents such as ethylenediaminetetraacetic acid (EDTA).

In certain embodiments, the functional ingredient is an additional hydration agent to the amino acid formulation. The additional hydration agent can be a synergist when combined with the amino acid formulation further enhances the hydration or rehydration effect of the composition. In one particular embodiment, the additional hydration agent is a carbohydrate to supplement energy stores burned by muscles. Suitable carbohydrates for use in particular embodiments of this disclosure are described in U.S. Pat. Nos. 4,312,856, 4,853,237, 5,681,569, and 6,989,171. Non-limiting examples of suitable carbohydrates include monosaccharides, disaccharides, oligosaccharides, complex polysaccharides or combinations thereof. Non-limiting examples of suitable types of monosaccharides for use in particular embodiments include trioses, tetroses, pentoses, hexoses, heptoses, octoses, and nonoses. Non-limiting examples of specific types of suitable monosaccharides include glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, and sialose. Non-limiting examples of suitable disaccharides include sucrose, lactose, and maltose. Non-limiting examples of suitable oligosaccharides include saccharose, maltotriose, and maltodextrin. In other particular embodiments, the carbohydrates are provided by a corn syrup, a beet sugar, a cane sugar, a juice, or a tea.

In another particular embodiment, the additional hydration agent is a flavanol that provides cellular rehydration. Flavanols are a class of natural substances present in plants, and generally comprise a 2-phenylbenzopyrone molecular skeleton attached to one or more chemical moieties. Non limiting examples of suitable flavanols for use in particular embodiments of this disclosure include catechin, epicatechin, gallocatechin, epigallocatechin, epicatechin gallate, epigallocatechin 3-gallate, theaflavin, theaflavin 3-gallate, theaflavin 3′-gallate, theaflavin 3,3′ gallate, thearubigin or combinations thereof. Several common sources of flavanols include tea plants, fruits, vegetables, and flowers. In preferred embodiments, the flavanol is extracted from green tea.

In a particular embodiment, the additional hydration agent is a glycerol solution to enhance exercise endurance. The ingestion of a glycerol containing solution has been shown to provide beneficial physiological effects, such as expanded blood volume, lower heart rate, and lower rectal temperature.

In certain embodiments, the functional ingredient is chosen from at least one probiotic, prebiotic and combination thereof. The probiotic is a beneficial microorganism that affects the human body's naturally-occurring gastrointestinal microflora. Examples of probiotics include, but are not limited to, bacteria of the genus Lactobacilli, Bifidobacteria, Streptococci, or combinations thereof, that confer beneficial effects to humans. In particular embodiments of the disclosure, the at least one probiotic is chosen from the genus Lactobacilli. According to other particular embodiments of this disclosure, the probiotic is chosen from the genus Bifidobacteria. In a particular embodiment, the probiotic is chosen from the genus Streptococcus.

Probiotics that may be used in accordance with this disclosure are well-known to those of skill in the art. Non-limiting examples of foodstuffs comprising probiotics include yogurt, sauerkraut, kefir, kimchi, fermented vegetables, and other foodstuffs containing a microbial element that beneficially affects the host animal by improving the intestinal microbalance.

Prebiotics, in accordance with the embodiments of this disclosure, include, without limitation, mucopolysaccharides, oligosacchaides, polysaccharides, amino acids, vitamins, nutrient precursors, proteins and combinations thereof. According to a particular embodiment of this disclosure, the prebiotic is chosen from dietary fibers, including, without limitation, polysaccharides and oligosaccharides. Non-limiting examples of oligosaccharides that are categorized as prebiotics in accordance with particular embodiments of this disclosure include fructooligosaccharides, inulins, isomalto-oligosaccharides, lactilol, lactosucrose, lactulose, pyrodextrins, soy oligosaccharides, transgalacto-oligosaccharides, and xylo-oligosaccharides. In other embodiments, the prebiotic is an amino acid. Although a number of known prebiotics break down to provide carbohydrates for probiotics, some probiotics also require amino acids for nourishment.

Prebiotics are found naturally in a variety of foods including, without limitation, bananas, berries, asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans).

In certain embodiments, the functional ingredient is at least one weight management agent. As used herein, “a weight management agent” includes an appetite suppressant and/or a thermogenesis agent. As used herein, the phrases “appetite suppressant”, “appetite satiation compositions”, “satiety agents”, and “satiety ingredients” are synonymous. The phrase “appetite suppressant” describes macronutrients, herbal extracts, exogenous hormones, anorectics, anorexigenics, pharmaceutical drugs, and combinations thereof, that when delivered in an effective amount, suppress, inhibit, reduce, or otherwise curtail a person's appetite. The phrase “thermogenesis agent” describes macronutrients, herbal extracts, exogenous hormones, anorectics, anorexigenics, pharmaceutical drugs, and combinations thereof, that when delivered in an effective amount, activate or otherwise enhance a person's thermogenesis or metabolism.

Suitable weight management agents include macronutrients selected from the group consisting of proteins, carbohydrates, dietary fats, and combinations thereof. Consumption of proteins, carbohydrates, and dietary fats stimulates the release of peptides with appetite suppressing effects. For example, consumption of proteins and dietary fats stimulates the release of the gut hormone cholecytokinin (CCK), while consumption of carbohydrates and dietary fats stimulates release of Glucagon-like peptide 1 (GLP-1).

Suitable macronutrient weight management agents also include carbohydrates. Carbohydrates generally comprise sugars, starches, cellulose and gums that the body converts into glucose for energy. Carbohydrates often are classified into two categories, digestible carbohydrates (e.g., monosaccharides, disaccharides, and starch) and non-digestible carbohydrates (e.g., dietary fiber). Studies have shown that non-digestible carbohydrates and complex polymeric carbohydrates having reduced absorption and digestibility in the small intestine stimulate physiologic responses that inhibit food intake. Accordingly, the carbohydrates embodied herein desirably comprise non-digestible carbohydrates or carbohydrates with reduced digestibility. Non-limiting examples of such carbohydrates include polydextrose; inulin; monosaccharide-derived polyols such as erythritol, mannitol, xylitol, and sorbitol; disaccharide-derived alcohols such as isomalt, lactitol, and maltitol; and hydrogenated starch hydrolysates. Carbohydrates are described in more detail herein below.

In another particular embodiment, the weight management agent is a dietary fat. Dietary fats are lipids comprising combinations of saturated and unsaturated fatty acids. Polyunsaturated fatty acids have been shown to have a greater satiating power than mono-unsaturated fatty acids. Accordingly, the dietary fats embodied herein desirably comprise poly-unsaturated fatty acids, non-limiting examples of which include triacylglycerols. In another particular embodiment, the weight management agent is an herbal extract. Extracts from numerous types of plants have been identified as possessing appetite suppressant properties. Non-limiting examples of plants whose extracts have appetite suppressant properties include plants of the genus Hoodia, Trichocaulon, Caralluma, Stapelia, Orbea, Asclepias, and Camelia. Other embodiments include extracts derived from Gymnema Sylvestre, Kola Nut, Citrus Aurantium, Yerba Mate, Griffonia Simplicifolia, Guarana, myrrh, guggul Lipid, and black current seed oil.

The herbal extracts may be prepared from any type of plant material or plant biomass. Non-limiting examples of plant material and biomass include the stems, roots, leaves, dried powder obtained from the plant material, and sap or dried sap. The herbal extracts generally are prepared by extracting sap from the plant and then spray-drying the sap. Alternatively, solvent extraction procedures may be employed. Following the initial extraction, it may be desirable to further fractionate the initial extract (e.g., by column chromatography) in order to obtain an herbal extract with enhanced activity. Such techniques are well known to those of ordinary skill in the art.

In one embodiment, the herbal extract is derived from a plant of the genus Hoodia. A sterol glycoside of Hoodia, known as P57, is believed to be responsible for the appetite-suppressant effect of the Hoodia species. In another embodiment, the herbal extract is derived from a plant of the genus Caralluma, non-limiting examples of which include caratuberside A, caratuberside B, bouceroside 1, bouceroside H, bouceroside III, bouceroside IV, bouceroside V, bouceroside VI, bouceroside VII, bouceroside VIII, bouceroside IX, and bouceroside X. In another embodiment, the at least one herbal extract is derived from a plant of the genus Trichocaulon. Trichocaulon plants are succulents that generally are native to southern Africa, similar to Hoodia, and include the species T. piliferum and T. officinale. In another embodiment, the herbal extract is derived from a plant of the genus Stapelia or Orbea. Not wishing to be bound by any theory, it is believed that the compounds exhibiting appetite suppressant activity are saponins, such as pregnane glycosides, which include stavarosides A, B, C, D, E, F, G, H, I, J, and K. In another embodiment, the herbal extract is derived from a plant of the genus Asclepias. Not wishing to be bound by any theory, it is believed that the extracts comprise steroidal compounds, such as pregnane glycosides and pregnane aglycone, having appetite suppressant effects. In another particular embodiment, the weight management agent is an exogenous hormone having a weight management effect Non-limiting examples of such hormones include CCK, peptide YY, ghrelin, bombesin and gastrin-releasing peptide (GRP), enterostatin, apolipoprotein A-IV, GLP-1, amylin, somastatin, and leptin.

In another embodiment, the weight management agent is a pharmaceutical drug. Non limiting examples include phentenime, diethylpropion, phendimetrazine, sibutramine, rimonabant, oxyntomodulin, floxetine hydrochloride, ephedrine, phenethylamine, or other stimulants.

In certain embodiments, the functional ingredient is at least one osteoporosis management agent. In certain embodiments, the osteoporosis management agent is at least one calcium source. According to a particular embodiment, the calcium source is any compound containing calcium, including salt complexes, solubilized species, and other forms of calcium. Non-limiting examples of calcium sources include amino acid chelated calcium, calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium citrate malate, calcium gluconate, calcium tartrate, calcium lactate, solubilized species thereof, and combinations thereof.

According to a particular embodiment, the osteoporosis management agent is a magnesium source. The magnesium source is any compound containing magnesium, including salt complexes, solubilized species, and other forms of magnesium. Non-limiting examples of magnesium sources include magnesium chloride, magnesium citrate, magnesium gluceptate, magnesium gluconate, magnesium lactate, magnesium hydroxide, magnesium picolate, magnesium sulfate, solubilized species thereof, and mixtures thereof. In another particular embodiment, the magnesium source comprises an amino acid chelated or amino acid formulation chelated magnesium.

In other embodiments, the osteoporosis agent is chosen from vitamins D, C, K, their precursors and/or beta-carotene and combinations thereof.

Numerous plants and plant extracts also have been identified as being effective in the prevention and treatment of osteoporosis. Non-limiting examples of suitable plants and plant extracts as osteoporosis management agents include species of the genus Taraxacum and Amelanchier, as disclosed in U.S. Patent Publication No. 2005/0106215, and species of the genus Lindera, Artemisia, Acorus, Carthamus, Carum, Cnidium, Curcuma, Cyperus, Juniperus, Prunus, Iris, Cichorium, Dodonaea, Epimedium, Erigonoum, rioya, Mentha, Ocimum, thymus, Tanacetum, Plantago, Spearmint, Bixa, Vitis, Rosemarinus, Rhus, and Anethum, as disclosed in U.S. Pat. App. No. 2005/0079232.

In certain embodiments, the functional ingredient is at least one phytoestrogen. Phytoestrogens are compounds found in plants which can typically be delivered into human bodies by ingestion of the plants or the plant parts having the phytoestrogens. As used herein, “phytoestrogen” refers to any substance which, when introduced into a body causes an estrogen like effect of any degree. For example, a phytoestrogen may bind to estrogen receptors within the body and have a small estrogen-like effect.

Examples of suitable phytoestrogens for embodiments of this disclosure include, but are not limited to, isoflavones, stilbenes, lignans, resorcyclic acid lactones, coumestans, coumestrol, equol, and combinations thereof. Sources of suitable phytoestrogens include, but are not limited to, whole grains, cereals, fibers, fruits, vegetables, black cohosh, agave root, black currant, black haw, chasteberries, cramp bark, dong quai root, devil's club root, false unicorn root, ginseng root, groundsel herb, licorice, liferoot herb, motherwort herb, peony root, raspberry leaves, rose family plants, sage leaves, sarsaparilla root, saw palmetto berried, wild yam root, yarrow blossoms, legumes, soybeans, soy products (e.g., miso, soy flour, soymilk, soy nuts, soy protein isolate, tempen, or tofu) chick peas, nuts, lentils, seeds, clover, red clover, dandelion leaves, dandelion roots, fenugreek seeds, green tea, hops, red wine, flaxseed, garlic, onions, linseed, borage, butterfly weed, caraway, chaste tree, vitex, dates, dill, fennel seed, gotu kola, milk thistle, pennyroyal, pomegranates, southernwood, soya flour, tansy, and root of the kudzu vine (pueraria root) and the like, and combinations thereof.

Isoflavones belong to the group of phytonutrients called polyphenols. In general, polyphenols (also known as “polyphenolics”), are a group of chemical substances found in plants, characterized by the presence of more than one phenol group per molecule.

Suitable phytoestrogen isoflavones in accordance with embodiments of this disclosure include genistein, daidzein, glycitein, biochanin A, formononetin, their respective naturally occurring glycosides and glycoside conjugates, matairesinol, secoisolariciresinol, enter olactone, enterodiol, textured vegetable protein, and combinations thereof. Suitable sources of isoflavones for embodiments of this disclosure include, but are not limited to, soy beans, soy products, legumes, alfalfa sprouts, chickpeas, peanuts, and red clover.

In certain embodiments, the functional ingredient is at least one long chain primary aliphatic saturated alcohol. Long-chain primary aliphatic saturated alcohols are a diverse group of organic compounds. The term alcohol refers to the fact these compounds feature a hydroxyl group (—OH) bound to a carbon atom. Non-limiting examples of particular long-chain primary aliphatic saturated alcohols for use in particular embodiments of the disclosure include the 8 carbon atom 1-octanol, the 9 carbon 1-nonanol, the 10 carbon atom 1-decanol, the 12 carbon atom 1-dodecanol, the 14 carbon atom 1-tetradecanol, the 16 carbon atom 1-hexadecanol, the 18 carbon atom 1-octadecanol, the 20 carbon atom 1-eicosanol, the 22 carbon 1-docosanol, the 24 carbon 1-tetracosanol, the 26 carbon 1-hexacosanol, the 27 carbon 1-heptacosanol, the 28 carbon 1-octanosol, the 29 carbon 1-nonacosanol, the 30 carbon 1-triacontanol, the 32 carbon 1-dotriacontanol, and the 34 carbon 1-tetracontanol.

In one embodiment, the long-chain primary aliphatic saturated alcohol is a policosanol. Policosanol is the term for a mixture of long-chain primary aliphatic saturated alcohols composed primarily of 28 carbon 1-octanosol and 30 carbon 1-triacontanol, as well as other alcohols in lower concentrations such as 22 carbon 1-docosanol, 24 carbon 1-tetracosanol, 26 carbon 1-hexacosanol, 27 carbon 1-heptacosanol, 29 carbon 1-nonacosanol, 32 carbon 1-dotriacontanol, and 34 carbon 1-tetracontanol.

In certain embodiments, the functional ingredient is at least one phytosterol, phytostanol or combination thereof. As used herein, the phrases “stanol”, “plant stanol” and “phytostanol” are synonymous. Plant sterols and stands are present naturally in small quantities in many fruits, vegetables, nuts, seeds, cereals, legumes, vegetable oils, bark of the trees and other plant sources Sterols are a subgroup of steroids with a hydroxyl group at C-3. Generally, phytosterols have a double bond within the steroid nucleus, like cholesterol, however, phytosterols also may comprise a substituted side chain (R) at C-24, such as an ethyl or methyl group, or an additional double bond. The structures of phytosterols are well known to those of skill in the art.

At least 44 naturally-occurring phytosterols have been discovered, and generally are derived from plants, such as corn, soy, wheat, and wood oils; however, they also may be produced synthetically to form compositions identical to those in nature or having properties similar to those of naturally-occurring phytosterols. Non-limiting suitable phytosterols include, but are not limited to, 4-desmethylsterols (e.g., b-sitosterol, campesterol, stigmasterol, brassicasterol, 22-dehydrobrassicasterol, and A5-avenasterol), 4-monomethyl sterols, and 4,4-dimethyl sterols (triterpene alcohols)(e.g., cycloartenol, 24-methylenecycloartanol, and cyclobranol).

As used herein, the phrases “stanol”, “plant stanol” and “phytostanol” are synonymous. Phytostanols are saturated sterol alcohols present in only trace amounts in nature and also may be synthetically produced, such as by hydrogenation of phytosterols. Suitable phytostanols include, but are not limited to, b-sitostanol, campestanol, cycloartanol, and saturated forms of other triterpene alcohols.

Both phytosterols and phytostanols, as used herein, include the various isomers such as the a and b isomers. The phytosterols and phytostanols of the present disclosure also may be in their ester form. Suitable methods for deriving the esters of phytosterols and phytostanols are well known to those of ordinary skill in the art, and are disclosed in U.S. Pat. Nos. 6,589,588, 6,635,774, 6,800,317, and U.S. Pat. App. No. 2003/0045473. Non limiting examples of suitable phytosterol and phytostanol esters include sitosterol acetate, sitosterol oleate, stigmasterol oleate, and their corresponding phytostanol esters. The phytosterols and phytostanols of the present disclosure also may include their derivatives.

Exemplary additives include, but not limited to, carbohydrates, polyols, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, plant extracts, flavonoids, alcohols, polymers and combinations thereof.

In one embodiment, the composition further comprises one or more polyols. The term “polyol”, as used herein, refers to a molecule that contains more than one hydroxyl group. A polyol may be a diol, triol, or a tetrad which contains 2, 3, and 4 hydroxyl groups respectively.

A polyol also may contain more than 4 hydroxyl groups, such as a pentad, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Non-limiting examples of polyols in some embodiments include maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, and sugar alcohols or any other carbohydrates capable of being reduced which do not adversely affect taste.

Suitable sugar acid additives include, but are not limited to, aldonic, uronic, aldaric, alginic, gluconic, glucuronic, glucaric, galactaric, galacturonic, and salts thereof (e.g., sodium, potassium, calcium, magnesium salts or other physiologically acceptable salts), and combinations thereof.

Suitable nucleotide additives include, but are not limited to, inosine monophosphate (IMP), guanosine monophosphate (GMP), adenosine monophosphate (AMP), cytosine monophosphate (CMP), uracil monophosphate (UMP), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g., guanine, cytosine, adenine, thymine, uracil).

Suitable organic acid additives include any compound which comprises a —COOH moiety, such as, for example, C2-C30 carboxylic acids, substituted hydroxyl C2-C30 carboxylic acids, butyric acid (ethyl esters), substituted butyric acid (ethyl esters), benzoic acid, substituted benzoic acids (e.g, 2,4-dihydroxybenzoic acid), substituted cinnamic acids, hydroxyacids, substituted hydroxybenzoic acids, anisic acid substituted cyclohexyl carboxylic acids, tannic acid, aconitic acid, lactic acid, tartaric acid, citric acid, isocitric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, amino acid formulation, caffeic acid, bile acids, acetic acid, ascorbic acid, alginic acid, erythorbic acid, polyglutamic acid, glucono delta lactone, and their alkali or alkaline earth metal salt derivatives thereof. In addition, the organic acid additives also may be in either the D- or L-configuration. Suitable organic acid additive salts include, but are not limited to, sodium, calcium, potassium, and magnesium salts of all organic acids, such as salts of citric acid, malic acid, tartaric acid, fumaric acid, lactic acid (e.g, sodium lactate), alginic acid (e.g, sodium alginate), ascorbic acid (e.g, sodium ascorbate), benzoic acid (e.g, sodium benzoate or potassium benzoate), sorbic acid and adipic acid. The examples of the organic acid additives described optionally may be substituted with at least one group chosen from hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfmyl, sulfamyl, carboxalkoxy, carboxamido, phosphonyl, phosphinyl, phosphoryl, phosphino, thioester, thioether, anhydride, oximino, hydrazino, carbamyl, phosphor or phosphonato. In particular embodiments, the organic acid additive is present in the sweetener composition in an amount effective to provide a concentration from about 10 ppm to about 5,000 ppm when present in a consumable, such as, for example, a beverage.

Suitable inorganic acid additives include, but are not limited to, phosphoric acid, phosphorous acid, polyphosphoric acid, hydrochloric acid, sulfuric acid, carbonic acid, sodium dihydrogen phosphate, and alkali or alkaline earth metal salts thereof (e.g, inositol hexaphosphate Mg/Ca).

Suitable bitter compound additives include, but are not limited to, caffeine, quinine, urea, bitter orange oil, naringin, quassia, and salts thereof.

Suitable flavorants and flavoring ingredient additives include, but are not limited to, vanillin, vanilla extract, mango extract, cinnamon, citrus, coconut, ginger, viridiflorol, almond, menthol (including menthol without mint), grape skin extract, and grape seed extract. “Flavorant” and “flavoring ingredient” are synonymous and can include natural or synthetic substances or combinations thereof. Flavorants also include any other substance which imparts flavor and may include natural or non-natural (synthetic) substances which are safe for human or animals when used in a generally accepted range. Non-limiting examples of proprietary flavorants include DOHLER™ Natural Flavoring Sweetness Enhancer K14323 (DOHLER™, Darmstadt, Germany), Symrise™ Natural Flavor Mask for Sweeteners 161453 and 164126 (SYMRISE™, Holzminden, Germany), Natural Advantage™ Bitterness Blockers 1, 2, 9 and 10 (Natural Advantage™, Freehold, New Jersey, U.S.A.), and SUCRAMASK™ (Creative Research Management, Stockton, California, U.S.A.). Suitable polymer additives include, but are not limited to, chitosan, pectin, pectic, pectinic, polyuronic, polygalacturonic acid, starch, food hydrocolloid or crude extracts thereof (e.g., gum acacia Senegal (FIBERGUM™), gum acacia seyal, carageenan), poly-L-lysine (e.g., poly-L-α-lysine or poly-L-ε-lysine), poly-L-ornithine (e.g., poly-L-α-omithine or poly-L-ε-ornithine), polypropylene glycol, polyethylene glycol, poly(ethylene glycol methyl ether), polyarginine, polyaspartic acid, polyglutamic acid, polyethylene imine, alginic acid, sodium alginate, propylene glycol alginate, and sodium polyethyleneglycolaiginate, sodium hexametaphosphate and its salts, and other cationic polymers and anionic polymers.

Suitable protein or protein hydrolysate additives include, but are not limited to, bovine serum albumin (BSA), whey protein (including fractions or concentrates thereof such as 90%, instant whey protein isolate, 34% whey protein, 50% hydrolyzed whey protein, and 80% whey protein concentrate), soluble rice protein, soy protein, protein isolates, protein hydrolysates, reaction products of protein hydrolysates, glycoproteins, and/or proteoglycans containing amino acids (e.g., glycine, alanine, serine, threonine, asparagine, glutamine, arginine, valine, isoleucine, leucine, norvaline, methionine, proline, tyrosine, hydroxyproline, and the like), collagen (e.g., gelatin), partially hydrolyzed collagen (e.g., hydrolyzed fish collagen), and collagen hydrolysates (e.g., porcine collagen hydrolysate).

In some example embodiments, the oral composition for hydration or rehydration may comprise protein hydrolysate additive. In related embodiments the composition may include protein hydrolysate additive as a replacement to the amino acid, such that the protein hydrolysate would be the sole source, if any, of amino acids in the composition. In other embodiments, at least one protein hydrolysate, such as whey protein, would include BCAA without the addition of other amino acids in the oral composition for hydration or rehydration formulation. In an alternative embodiments, the oral composition for hydration or rehydration comprises at least one protein hydrolysate, such as whey protein, that would include BCAA, but would also include the addition of other amino acids in the oral composition for hydration or rehydration formulation which would be of additive effect to the total percent of amino acid in the oral composition for hydration or rehydration formulation.

Suitable surfactant additives include, but are not limited to, polysorbates (e.g., polyoxyethylene sorbitan monooleate (polysorbate 80), polysorbate 20, polysorbate 60), sodium dodecylbenzenesulfonate, dioctyl sulfosuccinate or dioctyl sulfosuccinate sodium, sodium dodecyl sulfate, cetylpyridinium chloride (hexadecylpyridinium chloride), hexadecyltrimethylammonium bromide, sodium cholate, carbamoyl, choline chloride, sodium glycocholate, sodium taurodeoxycholate, lauric arginate, sodium stearoyl lactylate, sodium taurocholate, lecithins, sucrose oleate esters, sucrose stearate esters, sucrose palmitate esters, sucrose laurate esters, and other emulsifiers, and the like.

Suitable flavonoid additives are classified as flavonols, flavones, flavanones, flavan-3-ols, isoflavones, or anthocyanidins. Non-limiting examples of flavonoid additives include, but are not limited to, catechins (e.g., green tea extracts such as Polyphenon™ 60, Polyphenon™ 30, and Polyphenon™25 (Mitsui Norin Co., Ltd., Japan), polyphenols, rutins (e.g., enzyme modified rutin Sanmelin™ AO (San-fi Gen F.F.I., Inc., Osaka, Japan)), neohesperidin, naringin, neohesperidin dihydrochalcone, and the like. Suitable alcohol additives include, but are not limited to, ethanol.

Suitable astringent compound additives include, but are not limited to, tannic acid, europium chloride (EuCh), gadolinium chloride (GdCh), terbium chloride (TbCl₃), alum, tannic acid, and polyphenols (e.g., tea polyphenols).

### Compositions and Methods

The present composition may generally be in any edible form, such as liquid, semi-liquid, solid, or semi-solid. In some embodiments, the present composition is a beverage or beverage product. One example of the beverage is a ready-to-drink beverage. Ready-to-drink beverages include carbonated and non-carbonated beverages. Carbonated beverages include, but are not limited to, frozen carbonated beverages, enhanced sparkling beverages, cola, fruit-flavored sparkling beverages (e.g. lemon-lime, orange, grape, strawberry and pineapple), ginger-ale, soft drinks and root beer. Non-carbonated beverages include, but are not limited to, fruit juice, fruit-flavored juice, juice drinks, nectars, vegetable juice, vegetable-flavored juice, sports drinks, energy drinks, enhanced water drinks, enhanced water with vitamins, near water drinks (e.g., water with natural or synthetic flavorants), coconut water, tea type drinks (e.g. black tea, green tea, red tea, oolong tea), coffee, cocoa drink, beverage containing milk components (e.g. milk beverages, coffee containing milk components, cafe au lait, milk tea, fruit milk beverages), beverages containing cereal extracts and smoothies.

In a particular embodiment, the present disclosure relates to a sports drink or an enhanced water drink.

The beverage can be a full-calorie beverage that has up to about 120 calories per 8 oz Serving. The beverage can be a mid-calorie beverage that has up to about 60 calories per 8 oz. serving. The beverage can be a low-calorie beverage that has up to about 40 calories per 8 oz. serving. The beverage can be a zero-calorie that has less than about 5 calories per 8 oz. serving.

In another particular embodiment, the beverage does not contain milk and/or dairy components.

Beverages of the present disclosure exhibit minimal amino acid formulation degradation over a period of at least three months when stored at 5° C. In particular embodiments, the amino acid formulation concentration of the beverage after three months of storage at 5° C. is at least 90% of the initial amino acid formulation concentration, at least 95% of the initial amino acid formulation concentration, at least 97% of the initial amino acid formulation concentration, at least 98% of the initial amino acid formulation concentration, or at least 99% of the initial amino acid formulation concentration. “Initial concentration” refers to the concentration of amino acid formulation measured upon formulation, e.g. within 24 hours of preparing the beverage.

In other embodiments, the amino acid formulation concentration of the beverage is at least 90% of the initial amino acid formulation concentration when stored at 5° C. for four months, five months, six months, seven months, eight months, nine months, ten months, eleven months or twelve months.

In some embodiments, the present beverage has a plasma osmolality in a range from about 250 to about 350 mOsm/kg, or from about 270 to about 330 mOsm/kg, or from about 290 to about 310 mOsm/kg, or from about 290 to about 300 mOsm/kg.

The present disclosure also provides a method of preparing a ready-to-drink beverage comprising (i) providing a beverage matrix and (ii) adding the beverage ingredients described herein above to the beverage matrix, thereby providing a ready-to-drink beverage. The method optionally includes a further mixing step whereby the beverage ingredients and matrix are mixed to promote dissolution. The method can also optionally include a heating step, whereby the beverage ingredients and matrix are heated to promote dissolution.

Beverage ingredients are dissolved in the beverage matrix. Exemplary beverage matrices include water of beverage quality, for example tap water, deionized water, distilled water, reverse osmosis water, carbon-treated water, purified water, demineralized water and combinations thereof. Additional suitable matrices include, but are not limited to phosphoric acid, phosphate buffer, citric acid, citrate buffer and carbon-treated water.

The method can be performed at any temperature required to formulate the ready-to-drink beverage. For example, for ingredients that are temperature sensitive, the method is carried out below 70° C. Similarly, the beverage ingredients can be added to the beverage matrix in any order.

In some embodiments, the present composition is in a dry or semi-dry form such as a dry powder, a beverage concentrate, a tablet, a capsule, a gel, a gum. The dry composition can be readily consumable or be readily and rapidly soluble in a drinkable medium such as water.

In one particular embodiment, the present compositions may be prepared in concentrate or powder form to be reconstituted for use by the subject by the addition of water or any other appropriate liquid. Such reconstitution is made with the requisite amounts of water/liquid to ensure that the beverage to be consumed contains the active components in the proportions previously noted. In another embodiment, the composition may be solubilized in water/liquid and then brought to a frozen state, so as to provide, for example, flavored ices on sticks, like the ones known under the commercial name or trade mark “Popsicle.”

In one embodiment, a method for preparing a ready-to-drink solution comprising amino acid formulation comprises mixing an oral composition described herein with a drinkable medium in a weight ratio such that the concentration of amino acid formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L based on the total volume of the ready-to-drink solution. Exemplary drinkable media include water of beverage quality, for example tap water, deionized water, distilled water, reverse osmosis water, carbon-treated water, purified water, demineralized water, and combinations thereof. The drinkable medium may also be an existing beverage such as sparkling water, juice, tea, milk, or coffee.

In some embodiments, the present composition may be prepared as a dry powder mixture. The dry powder mixture is combined with a support for dispersion in water/liquid, such as maltodextrin, with a non-glucose sweet taste base, such as neohesperidine dihydrochalcone, at a level so as not to exceed 120 kcal/L, when reconstituted, and possibly flavored with fruit extracts or aromas, such as orange, lemon, strawberry, or others. One dose is intended for dissolution in 591 ml drinking water/liquid (one standard sports bottle). The concentrations are merely indicative, and more concentrated drinks may be prepared on the same formula basis.

The above powder mix may be added to a suitable support for tablet compression, with good organoleptic properties, such as sorbitol and magnesium stearate. The mass is possibly edulcorated with a known natural sweetener such as Neohesperidine dihydrochalcone and the total energy content should not exceed 120 kcal in the average daily intake. The product may be flavored with any choice of fruit or other flavors, such as orange, lemon, menthol, eucaplytol, or the like. The compressed tablets (or equivalent solid forms with the same composition) are intended for an average daily intake of between 5 and 10 tablets.

In some embodiments, the present disclosure relates to methods for hydration or rehydration. The method generally includes administering any oral composition described herein.

The present method generally utilizes the compositions described herein for at least one of the following purposes: providing a quick impact on plasma volume restoration during rehydration stage, attenuating or reversing the effects of dehydration or hypohydration, ameliorating other adverse effects of exercise, heat or other activity which causes bodily fluid loss, providing positive impact on following physical performance, enhancing the duration of body fluid retention, rapidly increasing the plasma volume, maintaining the increased plasma volume for long duration, restoring electrolyte balance, providing energy source, improving physical performance, limiting or reducing calorie uptake.

In some embodiments, the present disclosure provides a method of hydrating or rehydrating a human, the method comprising administering an effective amount of the oral composition described herein, wherein the oral composition comprises an amino acid formulation described herein. Administration of the oral composition can be before, during, or after dehydration or loss of body fluid.

In some embodiments, the method comprises administering a sports beverage to an human described herein, wherein the sports drink is further configured to enhance sports performance of the human, reduce lactate production, reduce perceived exhaustion, reduce muscle soreness (both actual muscle damage and perceived soreness), improve time to exhaustion, improve time trial performance, improve power output, reduce production of lactic acid, and reduce net fluid loss observed with exercise. As used herein, “enhanced sports performance” refers to an improvement in sports performance associated with consuming embodiments of the sports beverages provided herein, as compared to sports performance without consuming the sports beverage or water. The sports beverage may be consumed before, during, or after sports performance. As used herein, “sports performance” refers to both endurance exercise and non-endurance exercise. Endurance exercise includes aerobic activities over prolonged periods of time (e.g., greater than about 30 minutes) while non-endurance exercise includes aerobic activities over a shorter period of time (e.g., less than about 30 minutes).

In one particular embodiment, the present disclosure relates to a method of enhancing athletic performance comprising administering/consuming a sports beverage before, during, or after endurance exercise, wherein the sports beverage comprises an aqueous solution of an amino acid formulation described herein, wherein the amino acid formulation is in an amount from about 1 g/L to about 50 g/L, or from about 2 g/L to about 30 g/L, or from about 3 g/L to about 30 g/L, or from about 4 g/L to about 20 g/L, or from about 5 g/L, to about 10 g/L, or from about 0.05 g/L to about 1 g/L, based on the total volume of the sports beverage. In one embodiment, the amino acid formulation consists essentially of glycine. In one embodiment, the sports drink comprises glycine as the only amino acid and is free or substantially free from other amino acid. In another particular embodiment, the amino acid formulation consists essentially of glycine, or a glycine-containing dipeptide, or a carbohydrate-glycine complex, or any combinations thereof.

In some embodiments, the method include preparing a drinkable solution by dissolving a dry powder composition comprising the amino acid formulation in a drinkable medium comprising water, and administering the drinkable solution orally. In some embodiments, the concentration of the amino acid formulation is from about 1 g/L to about 50 g/L, or from about 2 g/L to about 30 g/L, or from about 3 g/L to about WL, or from about 4 g/L to about 20 g/L, or from about 5 g/L to about 10 g/L, or from about 0.05 g/L to about 1 g/L, based on the total volume of the drinkable solution.

In other embodiments, the method include consuming/ingesting the oral composition and a drinkable medium comprising water. Ingestion of the oral composition and a drinkable medium can be concurrently, simultaneously, separately, or successively. In some embodiments, the ratio of the oral composition to the drinkable medium is such that the content of the amino acid formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, relative to the total volume of the drinkable medium.

The methods according to the present disclosure provide rapid and effective hydration and retention of body fluid of a human. In some embodiments, the plasma volume of the human is increased by at least about 2.5%, or at least about 3%, or at least about 4% in about 10 minutes after administration of the oral composition.

In some embodiments, the plasma volume of the human is increased by at least about 4.5%, or at least about 5%, or at least about 5.5%, or at least about 6% in about 15 minutes after administration of the oral composition.

In some embodiments, the plasma volume of the human is increased by at least about 5%, or at least about 5.5%, or at least about 6%, or at least about 6.5%, or at least about 7% in about 30 minutes after administration of the oral composition.

In some embodiments, the plasma volume of the human is increased by at least about 3%, or at least about 3.5%, or at least about 4%, or at least about 5%, or at least about 6% in about 45 minutes after administration of the oral composition.

In some embodiments, the plasma volume of the human is increased by at least about 3%, or at least about 3.5%, or at least about 4%, or at least about 5%, or at least about 6% on in about 60 minutes after administration of the oral composition.

In some embodiments, the plasma volume of the human measured at 30 minutes after administration of the oral composition remains substantially unchanged thereafter for at least about 15 minutes, or at least about 30 minutes, or at least about 1 hour.

In some embodiments, the plasma volume of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.

In some embodiments of the present method, the plasma osmolality of the human is maintained in a range from about 270 to about 330 mOsm/kg, or from about 280 to about 320 mOsm/kg, or from about 290 to about 310 mOsm/kg, or from about 290 to about 300 mOsm/kg, in at least 60 minutes after administration of the oral composition.

In some embodiments, the change of the plasma osmolality of the human is no greater than 3 mOsm/kg in at least 60 minutes after administration of the oral composition.

The following examples illustrate preferred, but non-limiting embodiments of the present disclosure.

### EXAMPLES

### Example 1—Beverages Comprising Amino Acid Formulations and Rehydration Test

Three example beverages were prepared by mixing amino acid formulation with water. Bev 780 comprises glycine in an amount of about 9.14 g/L in water. Bev 236 comprises L-alanine in an amount of about 5.96 g/L in water. Bev 588 is a control beverage with no added amino acid.

Fifteen volunteers were subjected to the rehydration test. The test was a randomized, double-blind, crossover study with healthy participants following the protocol similar to that described by Shirreffs et al. (Med Sci Sports Exerc, 1996). The volunteers were first subject to an Exercise-Heat session and then each was instructed to consume the three beverages prepared above for a following rehydration test Each volunteer completed one trial for each test beverage, resulting in a total of three trials per volunteer. The volunteers were first dehydrated and then each was instructed to consume one of the three beverages prepared above.

Exercise-Heat Session: During the exercise-heat/dehydration sessions, all proper safety precautions monitoring heart rate, core temperature were employed during the exercise bout in the heat (as required in all of our previous studies). In each experiment, following the point of eliciting dehydration of ˜1.5% body mass, subjects were instructed to begin the rehydration phase following a cool-down (ice sheet, air-conditioned room and brief shower). The fluid deficit incurred by the dehydration protocols was calculated from the acute change in body mass from pre- (baseline value calculated from mean of days 1, 2 and 3) to post- exercise/heat exposure(corrected for any food, fluid consumption, urine or feces loss) and expressed as a percent from baseline body mass (Cheuvront et. al. 2004, 2010). For all calculations water and body mass losses from sweat and urine was considered equivalent (1 L=1 kg). Sweat electrolytes were estimated by using the Brisson method (scapular pouch) to collect sweat at the end of each intermittent bout. Alternatively, on occasions cycling prohibited collection, we utilized the Tegaderm patch method (Baker, GSSI 2017) on the forearm to estimate losses of sodium, potassium, and chloride when the patch appeared saturated (approximately at 1.5% body mass loss). The Horiba Laqua-twin analyzers were used to measure sweat electrolyte losses.

Rehydration test Following Exercise-Heat Session: The procedures for rehydration sessions were a modification of the procedures employed by Shirreffs et al. (Med Sci Sports Exerc, 1996). After achieving the target body mass loss, the subjects were instructed to remain seated in a rest room at a temperate of about 20° C. to about 22° C. A venous blood sample from each tested subject was obtained after a 30 min seated rest period. The rehydration period were about 3 hour total duration after drinking the first bolus (125% fluid deficit was replaced by three equal volumes every 10 min) Subsequent measures were then obtained at 15, 30, 60, 90, and thereafter 120 and 180 min (for urine/body mass only) of recovery (similar to Shirreffs et al. 1996). Urine output and body mass were assessed every hour over 3 hours (following initial ingestion of compositions) to determine the cumulative urine mass and urine osmolality. Whole blood were analyzed for hemoglobin, hematocrit, and plasma for osmolality.

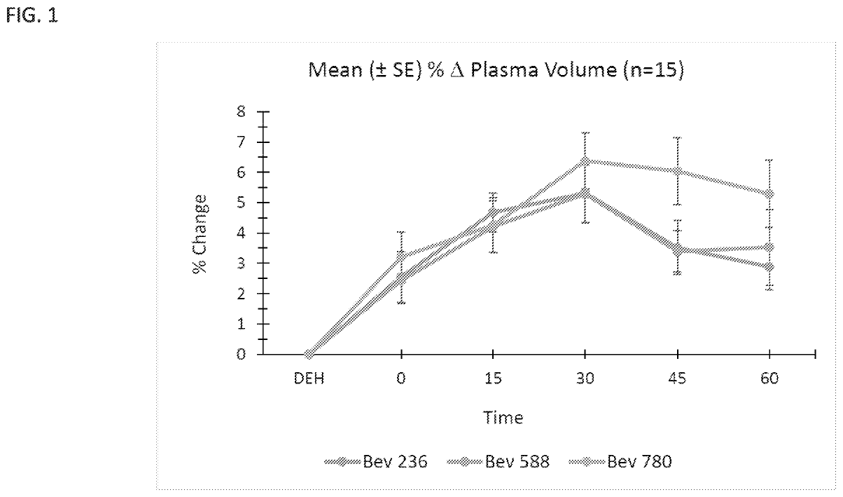

Plasma volume change over time after administration of the beverage was measured and summarized in FIG. 1. As can be seen, consumption of Bev 780 having about 9.14 g/L of glycine could rapidly increase the plasma volume by at least 3% at time zero, by at least 4% after 15 minutes, at least 6% after 30 minutes. The plasma volume measured at 30 minutes after consumption did not show significant change thereafter, and the plasma volume was found to remain at an increased level at least 5% higher compared to the level prior to rehydration Bev 236 comprising about 5.96 g/L. of alanine also showed rapid increase of plasma volume in 30 minutes after consumption. Bev 780 tended to be higher in the total plasma volume increase compared to the control (Bev 588) at 45 minutes. Bev 780 was also found to be higher in the total plasma volume increase compared with Bev 236 at 60 minutes. Compared with the control beverage (Bev 588), the beverages comprising amino acid formulations clearly showed the significant improvement in the retention of plasma volume. These results further support the rehydration effectiveness of amino acids.

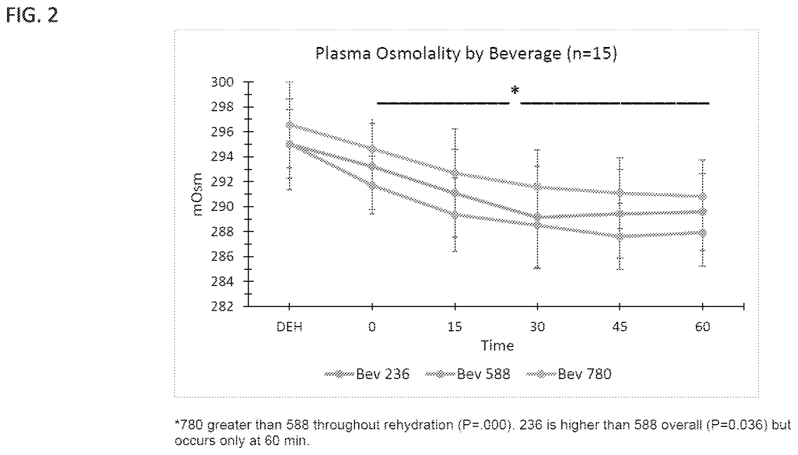

Plasma osmolality change of the tested volunteers over time after consumption of the beverages was also measured, and the results are summarized in FIG. 2. As can been seen, there was a significant overall beverage effect (p=0.001). Plasma osmolality was significantly higher overall (by about 3 mosmol) through the 60 minutes following rehydration with respect to Bev 780. Further, Bev 236 was higher (by about 1.6 mosmol) compared with the control beverage 588 (p=0.03). There was no significant difference between Bev 236 and Bev 780 (p=0.09), although Bev 780 tended to be higher than Bev 236 in the total plasma osmolality. The only time point during rehydration that Bev 236 was different from Bev 588 was at 60 minutes, while Bev 780 was higher than Bev 588 from immediately after rehydration onward (0 through 60 minutes). The higher plasma osmolality (FIG. 2) combined with higher total plasma volume (FIG. 1) further support improved restoration and retention of critical elements such as electrolytes after consuming the present compositions. Further, the higher osmolality caused by the amino acid containing beverages was found to effectively stimulate the thirst and stimulate the drinking need of the tested volunteers.

### NON-PATENT REFERENCES

Siamak, A. Adibi, Leucine absorption rate and net movements of sodium and water in human jejunum, Journal of Applied Physiology, 1970, 28(6), 753-757
M. D. Hellier, C. Thirumalai, C. D. Holdsworth, The effect of amino acids and dipeptides on sodium and water absorption in man, Gut, 1973, 14, 41-45.
D. B. A. Silk, P. D. Fairclough, N. J. Park, A. E. Lane, J. P. W. Webb, M. L. Clark, A. M. Dawson, A study of relations between the absorption of amino acids, dipeptides, water and electrolytes in the normal human jejunum, Clinical Science and Moleculir Medicine, 1975, 49, 401-408.
J. E. Hegarty, P. D. Fairclough, M. L. Clark, A. M. Dawson, Jejunal water and electrolyte secretion induced by L-arginine in man, Gut, 1981, 22, 108-113.
Kazunobu Okazaki, Yoshi-Ichiro Kamijo, Yoshiaki Takeno, Tadashi Okumoto, Shizue Masuki, Hiroshi Nose, Effects of exercise training on thermoregulatory responses and blood volume in older men, Journal of Applied Physiology, 2002, 93, 1630-1637.
Moise Coeffier, Bernadette Hecketsweiler, Philippe Hecketsweiler, and Pierre Dechelotte, Effect of glutamine on water and sodium absorption in human jejunum at baseline and during PGE₁-induced secretion, Journal of Applied Physiolgy, 2005, 98, 2163-2168.
Chih-Yin Tai, Jordan M Joy, Paul H Falcone, Laura R Carson, Matt M Mosman, Justen L Straight, Susie L Oury, Carlos Mendez Jr, Nick J Loveridge, Michael P Kim & Jordan R Moon, An amino acid-electrolyte beverage may increase cellular rehydration relative to carbohydrate-electrolyte and flavored water beverages. Nutrition Journal, 2014 (13), 47.
Kurt J Sollanek, Matthew Tsurumoto, Sadasivan Vidyasagar, Robert W Kenefick, Samuel N Cheuvront, Neither body mass nor sex influences beverage hydration index outcomes during randomized trial when comparing 3 commercial beverages. The American Journal of Clinical Nutrition, 2018, 107(4), 544-549.
Samuel N. Cheuvront, Robert W. Kenefick, Nisha Charkoudian, Katherine M. Mitchell, Adam J. Luippold, Karleigh E. Bradbury, Sadasivan Vidyasagar, Efficacy of Glucose or Amino Acid-Based Commercial Beverages in Meeting Oral Rehydration Therapy Goals After Acute Hypertonic and Isotonic Dehydration, J Parenter Enteral Nutr, 2018, 42(7), 1185-1193.
S. M. Shirreffs, A. J. Taylor, J. B. Leiper, R. J. Maughan, Post-exercise rehydration in man: effects of volume consumed and drink sodium content. Medicine and Science in Sports and Exercise, 1996, 28(10):1260-1271.
S. N Cheuvront, R. Carter 3rd, S. J. Montain, M. N. Sawka, Daily body mass variability and stability in active men undergoing exercise-heat stress. Int J Sport Nutr Exerc Metab. 2004, 14(5):532-40.
S. N. Cheuvront, B. R. Ely, R. W. Kenefick, M. N. Sawka, Biological variation and diagnostic accuracy of dehydration assessment markers. Am J Clin Nutr. 2010, 92(3):565-73.
L. B. Baker, Sweating Rate and Sweat Sodium Concentration in Athletes: A Review of Methodology and Intra/Interindividual Variability. Sports Med. 2017, 47(Suppl 1):111-128.

The following numbered clauses define further example aspects and features of the present disclosure:
1. An oral composition for hydration or rehydration, the oral composition comprising an amino acid formulation.
2. The oral composition of clause 1, wherein the amino acid formulation comprises a compound selected from the group consisting of an amino acid, a dipeptide, a carbohydrate-amino acid complex, or any combination thereof.
3. The oral composition of any one of clauses 1-2, wherein the amino acid is selected from the group consisting of leucine, isoleucine, valine, histidine, lysine, methionine, phenylalanine, threonine, tryptophan, alanine, arginine, asparagine, aspartic acid, cysteine, glutamic acid, glutamine, glycine, proline, serine, and tyrosine, or any combination thereof.
4. The oral composition of any one of clauses 1-3, wherein the dipeptide is selected from the group consisting of glycine-containing dipeptide, lysine-containing dipeptide, arginine-containing dipeptide, alanine-containing dipeptide, glutamine-containing dipeptide, or any combination thereof.
5. The oral composition of any one of clauses 1-4, wherein the carbohydrate-amino acid complex is selected from the group consisting of glutamine-glucose, alanine-glucose, or both.
6. The oral composition of clause 1, wherein the amino acid formulation consists essentially of glycine, or a glycine-containing dipeptide, or both.
7. The oral composition of any one of clauses 1-6, wherein the oral composition is a ready-to-drink hydrating beverage.
8. The oral composition of clause 7, wherein the concentration of the amino acid formulation is from about 1 g/L to about 50 g/L, or from about 2 g/L to about 30 g/L, or from about 3 g/L., to about 30 g/L, or from about 4 g/L to about 20 g/L, or from about 5 g/L to about 10 g/L, or from about 0.05 g/L to about 1 g/L, based on the total volume of the oral composition.
9. The oral composition of any one of clauses 1-6, wherein the oral composition is in a dry or semi-dry form.
10. The oral composition of clause 9, wherein the oral composition is a gum, tablet, capsule, or dry powder.
11. An oral composition being a ready-to-drink hydrating beverage, wherein the oral composition comprise an amino acid formulation, wherein the amino acid formulation consists essentially of glycine, and wherein the glycine concentration is from about 4 g/L to about 20 g/L, based on the total volume of the oral composition.
12. The oral composition of any one of clauses 1-11, wherein the amino acid formulation or the oral composition is free or substantially free from a branched-chain amino acid (BCAA).
13. The oral composition of any one of clauses 1-12, wherein the BCAA is selected from the group consisting of leucine, isoleucine, valine, and combinations thereof.
14. The oral composition of any one of clauses 1-13, further comprising at least one electrolyte selected from the group consisting of sodium, potassium, calcium, magnesium, chloride, phosphate, bicarbonate, and combinations thereof.
15. The oral composition of clause 14, wherein the total electrolyte concentration is at least about 200 mg/L.
16. The oral composition of any one of clauses 1-15, further comprising at least one sweetener.
17. The oral composition of clause 16, wherein the sweetener is selected from the group comprising stevia and steviol glycosides, Luo Han Guo and the related mogroside compounds, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols such as erythritol, sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs), and combinations thereof.
18. The oral composition of any one of clauses 1-17, further comprising at least one additive.
19. The oral composition of any one of clauses 1-18, further comprising at least one functional ingredient.
20. The oral composition of any one of clauses 1-19, wherein the oral composition is selected from a sports drink and an enhanced water drink.
21. The oral composition of any one of clauses 1-20, wherein the oral composition is selected from a full-calorie beverage, a mid-calorie beverage, a low-calorie beverage, or a zero-calorie beverage.
22. A method of hydrating or rehydrating a human, the method comprising administering an oral composition, the oral composition comprising an amino acid formulation.
23. The method of clause 22, wherein the amino acid formulation comprises a compound selected from the group consisting of an amino acid, a dipeptide, a carbohydrate-amino acid complex, or any combination thereof.
24. The method of any one of clauses 22-23, wherein the amino acid is selected from the group consisting of leucine, isoleucine, valine, histidine, lysine, methionine, phenylalanine, threonine, tryptophan, alanine, arginine, asparagine, aspartic acid, cysteine, glutamic acid, glutamine, glycine, proline, serine, and tyrosine, or any combination thereof.
25. The method of any one of clauses 22-24, wherein the dipeptide is selected from the group consisting of glycine-containing dipeptide, lysine-containing dipeptide, arginine-containing dipeptide, alanine-containing dipeptide, glutamine-containing dipeptide, or any combination thereof.
26. The method of any one of clauses 22-25, wherein the carbohydrate-amino acid complex is selected from the group consisting of glutamine-glucose, alanine-glucose, or both.
27. The method of clause 22, wherein the amino acid formulation consists essentially of glycine, or a glycine-containing dipeptide, or both.
28. The method of any one of clauses 22-27, wherein the oral composition is a ready-to-drink hydrating beverage, or a sports drink, or an enhanced water drink.
29. The method of any one of clauses 22-28, wherein the oral composition is selected from a full-calorie beverage, a mid-calorie beverage, a low-calorie beverage, or a zero-calorie beverage.
30. The method of any one of clauses 22-29, wherein the concentration of the amino acid formulation is from about 1 g/L to about 50 g/L., or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, based on the total volume of the oral composition.
31. The method of clause 22, wherein the oral composition is a ready-to-drink hydrating beverage, wherein the amino acid formulation consists essentially of glycine, and wherein the glycine concentration is from about 4 g/L to about 20 g/L based on the total volume of the beverage.
32. The method of any one of clauses 22-27, wherein the oral composition is in a dry or semi-dry form.
33. The method of clause 32, wherein the oral composition is a gum, tablet, capsule, or dry powder.
34. The method of any one of clauses 22-33, wherein the amino acid formulation or the oral composition is substantially free from a branched-chain amino acid (BCAA).
35. The method of any one of clauses 22-34, wherein the BCAA is selected from the group consisting of leucine, isoleucine, valine, and combinations thereof.
36. The method of any one of clauses 22-35, wherein the oral composition further comprises at least one electrolyte selected from the group consisting of sodium, potassium, calcium, magnesium, chloride, phosphate, bicarbonate, and combinations thereof.
37. The method of clause 36, wherein the total electrolyte concentration is at least about 200 mg/L.
38. The method of any one of clauses 22-37, wherein the oral composition further comprises at least one sweetener.
39. The method of any one of clauses 22-38, wherein the oral composition further comprises at least one additive.
40. The method of any one of clauses 22-39, wherein the oral composition further comprises at least one functional ingredient.
41. The method of any one of clauses 22-40, wherein the plasma volume of the human is increased by at least about 2.5%, or at least about 3%, or at least about 4% on or before 5 minutes after administration of the oral composition.
42. The method of any one of clauses 22-41, wherein the plasma volume of the human is increased by at least about 4.5%, or at least about 5%, or at least about 5.5%, or at least about 6% on or before 15 minutes after administration of the oral composition.
43. The method of any one of clauses 22-42, wherein the plasma volume of the human is increased by at least about 5%, or at least about 5.5%, or at least about 6%, or at least about 6.5%, or at least about 7% on or before 30 minutes after administration of the oral composition.
44. The method of any one of clauses 22-43, wherein the plasma volume of the human is increased by at least about 3%, or at least about 3.5%, or at least about 4%, or at least about 5%, or at least about 6% on or before 45 minutes after administration of the oral composition.
45. The method of any one of clauses 22-44, wherein the plasma volume of the human is increased by at least about 3%, or at least about 3.5%, or at least about 4%, or at least about 5%, or at least about 6% on or before 60 minutes after administration of the oral composition.
46. The method of any one of clauses 22-45, wherein the plasma volume of the human measured at 30 minutes after administration of the oral composition remains substantially unchanged thereafter for at least about 15 minutes, or at least about 30 minutes, or at least about 1 hour.
47. The method of any one of clauses 22-46, wherein the plasma volume of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.
48. The method of any one of clauses 22-47, wherein plasma osmolality of the human is maintained in a range from about 270 to about 330 mOsm/kg, or from about 280 to about 320 mOsm/kg, or from about 290 to about 310 mOsm/kg, or from about 290 to about 300 mOsm/kg, in at least 60 minutes after administration of the oral composition.
49. The method of any one of clauses 22-47, wherein plasma osmolality of the human prior to administration of the oral composition is changed by no greater than 3 mOsm/kg in at least 60 minutes after administration of the oral composition.
50. A method for improving physical or sports performance, the method comprising administering any oral composition according to clauses 1-21 and 53.
51. The method of clause 50, wherein the oral composition is administered before, during, or after endurance exercise.
52. The method of any of clauses 50-51, wherein the enhanced performance is characterized by reduced perceived physical exhaustion, reduced muscle soreness, reduced muscle damage, reduced net fluid loss, reduced production of lactic acid, improved time to exhaustion, improved time trial performance, improved power output or any combinations thereof.
53. The oral composition of any one of clauses 1-11 and 14-21, wherein the oral composition comprises at least one essential amino acid and at least one non-essential amino acid 54. The method of any one of clauses 22-33 and 36-52, wherein the oral composition comprises at least one essential amino acid and at least one non-essential amino acid.

All publications, patents and patent applications mentioned in the specification are indicative of the level of those skilled in the art to which this disclosure pertains.

All of the compositions and methods disclosed and claimed herein can be made and executed without undue experimentation in light of the present disclosure. While the compositions and methods of this disclosure have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the disclosure. More specifically, it will be apparent that certain agents, additives, and ingredients that are similar according to their physical, chemical, physiological, and/or gustative properties may be substituted for the agents, additives and ingredients described herein while the same or similar results would be achieved. All such similar substitutes and modifications apparent to those skilled in the art are deemed to be within the spirit, scope, and concept of the disclosure as defined by the hereinafter appended claims.

What is claimed is:

1. An oral composition for hydration or rehydration, the oral composition comprising an amino acid formulation.

2. The oral composition of claim 1, wherein the amino acid formulation comprises a compound selected from the group consisting of an amino acid, a dipeptide, a carbohydrate-amino acid complex, or any combination thereof.

3. The oral composition of any one of claims 1-2, wherein the amino acid is selected from the group consisting of leucine, isoleucine, valine, histidine, lysine, methionine, phenylalanine, threonine, tryptophan, alanine, arginine, asparagine, aspartic acid, cysteine, glutamic acid, glutamine, glycine, proline, serine, and tyrosine, or any combination thereof.

4. The oral composition of any one of claims 1-3, wherein the dipeptide is selected from the group consisting of glycine-containing dipeptide, lysine-containing dipeptide, arginine-containing dipeptide, alanine-containing dipeptide, glutamine-containing dipeptide, or any combination thereof.

5. The oral composition of any one of claims 1-4, wherein the carbohydrate-amino acid complex is selected from the group consisting of glutamine-glucose, alanine-glucose, or both.

6. The oral composition of claim 1, wherein the amino acid formulation consists essentially of glycine, or a glycine-containing dipeptide, or both.

7. The oral composition of any one of claims 1-6, wherein the oral composition is a ready-to-drink hydrating beverage.

8. The oral composition of claim 7, wherein the concentration of the amino acid formulation is from about 1 g/L to about 50 g/L, or from about 2 g/L to about 30 g/L, or from about 3 g/L to about 30 g/L, or from about 4 g/L to about 20 g/L, or from about 5 g/L to about 10 g/L, or from about 0.05 g/L to about 1 g/L, based on the total volume of the oral composition.

9. The oral composition of any one of claims 1-6, wherein the oral composition is in a dry or semi-dry form.

10. The oral composition of claim 9, wherein the oral composition is a gum, tablet, capsule, or dry powder.

11. A method of hydrating or rehydrating a human, the method comprising administering an oral composition, the oral composition comprising an amino acid formulation.

12. The method of claim 11, wherein the amino acid formulation comprises a compound selected from the group consisting of an amino acid, a dipeptide, a carbohydrate-amino acid complex, or any combination thereof.

13. The method of any one of claims 11-12, wherein the amino acid is selected from the group consisting of leucine, isoleucine, valine, histidine, lysine, methionine, phenylalanine, threonine, tryptophan, alanine, arginine, asparagine, aspartic acid, cysteine, glutamic acid, glutamine, glycine, proline, serine, and tyrosine, or any combination thereof.

14. The method of any one of claims 11-13, wherein the dipeptide is selected from the group consisting of glycine-containing dipeptide, lysine-containing dipeptide, arginine-containing dipeptide, alanine-containing dipeptide, glutamine-containing dipeptide, or any combination thereof.

15. The method of any one of claims 11-14, wherein the carbohydrate-amino acid complex is selected from the group consisting of glutamine-glucose, alanine-glucose, or both.

16. The method of any one of claims 11-15, wherein the plasma volume of the human is increased by at least about 2.5%, or at least about 3%, or at least about 4% on or before 5 minutes after administration of the oral composition.

17. The method of any one of claims 11-16, wherein the plasma volume of the human measured at 30 minutes after administration of the oral composition is decreased by less than about 1%, or less than about 2%, or less than about 3%, or less than about 4%, or less than about 5%, or less than about 6%, or less than about 7%, or less than about 8%, or less than about 9%, or less than about 10% for at least about 30 minutes.

18. The method of any one of claims 11-17, wherein plasma osmolality of the human is maintained in a range from about 270 to about 330 mOsm/kg, or from about 280 to about 320 mOsm/kg, or from about 290 to about 310 mOsm/kg, or from about 290 to about 300 mOsm/kg, in at least 60 minutes after administration of the oral composition.

19. A method for improving physical or sports performance, the method comprising administering any oral composition according to claims 1-10.

20. The oral composition of any one of claims 1-10, wherein the oral composition comprises at least one essential amino acid and at least one non-essential amino acid.

21. The method of any one of claims 11-19, wherein the oral composition comprises at least one essential amino acid and at least one non-essential amino acid.
